(19) United States
(12) Patent Application Publication
(10) Pub. No.: US 2010/0310623 A1
(43) Pub. Date: Dec. 9, 2010

(54) Synergetic functionalized spiral-in-tubular bone scaffolds

(76) Inventors: Cato T. Laurencin, Avon, CT (US); Xiaojun Yu, Fishers, IN (US); Chandra M. Valmikinathan, Elmwood Park, NJ (US); Junping Wang, Kearny, NJ (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/455,778

(22) Filed: Jun. 5, 2009

(51) Int. Cl. A61F 2/28; A61K 38/18; A61P 19/00

(52) U.S. Cl. 424/423; 514/7.6; 514/16.7

(57) ABSTRACT

An integrated scaffold for bone tissue engineering has a tubular outer shell and a spiral scaffold made of a porous sheet. The spiral scaffold is formed such that the porous sheet defines a series of spiral coils with gaps of controlled width between the coils to provide an open geometry for enhanced cell growth. The spiral scaffold resides within the bore of the shell and is integrated with the shell to fix the geometry of the spiral scaffold. Nanofibers may be deposited on the porous sheet to enhance cell penetration into the spiral scaffold. The spiral scaffold may have alternating layers of polymer and ceramic on the porous sheet that have been built up using a layer-by-layer method. The spiral scaffold may be seeded with cells by growing a cell sheet and placing the cell sheet on the porous sheet before it is rolled.

### FIELD OF THE INVENTION

The present invention relates to tissue engineered scaffolds for the repair of bone defects and techniques for fabricating three-dimensional tissues for transplantation in human recipients.

### BACKGROUND

### Tissue Scaffolds

The process of repair or replacement of whole tissues, or portions thereof, often involves a combination of cells, engineered scaffolds, suitable biochemical and physiochemical factors, and growth promoting proteins. Each tissue type requires unique mechanical and structural properties for proper functioning. During tissue repair or replacement, cells often are implanted or “seeded” into an artificial structure capable of supporting a three-dimensional tissue formation. These structures (“scaffolds”) often are critical to replicating the in vivo milieu and allowing the cells to influence their own microenvironment. Scaffolds may serve to allow cell attachment and migration, deliver and retain cells and biochemical factors, enable diffusion of vital cell nutrients and expressed products, and exert certain mechanical and biological influences to modify the behavior of the cell phase. A scaffold utilized with tissue reconstruction has several requisites. A scaffold should have a high porosity and an adequate pore size to facilitate cell seeding and diffusion of both cells and nutrients throughout the whole structure. Biodegradability of the scaffold is also an essential requisite. Scaffolds should be absorbed by the surrounding tissues without the necessity of a surgical removal. The rate at which degradation occurs has to coincide as much as possible with the rate of tissue formation. As cells are fabricating their own natural matrix structure around themselves, the scaffold provides structural integrity within the body and eventually degrades leaving the neotissue (newly formed tissue) to assume the mechanical load.

### Tissue Scaffold Materials

Several different materials (natural and synthetic, biodegradable and permanent) have been examined for use with scaffolds. Many of these materials, such as bioresorbable sutures, collagen, and some linear aliphatic polyesters, have been studied. Biomaterials have been engineered to incorporate additional features such as injectability, synthetic manufacture, biocompatibility, non-immunogenicity, transparency, nanoscale fibers, low concentration, and resorption rates.

Scaffolds may be constructed from synthetic materials, such as polylactic acid (PLA). PLA is a polyester which degrades within the human body to form a lactic acid byproduct which then is easily eliminated. Similar materials include polyglycolic acid (PGA) and polycaprolactone (PCL); they exhibit a faster and a slower rate, respectively, of degradation to lactic acid compared to PLA.

Scaffolds also may be constructed from natural materials. Several components of the extracellular matrix have been studied to evaluate their ability to support cell growth. Protein-based materials, such as collagen or fibrin, and polysaccharidic materials, such as chitosan or glycosaminoglycans (GAGs), have proved suitable in terms of cell compatibility. However, there are some concerns with potential immunogenicity.

An ideal bone tissue-engineered scaffold provides a three-dimensional matrix with high mechanical strength adequate to support the newly formed tissue, high porosities allowing the new tissue formation and growth within the scaffolds, biomimetic structure for nutrient transport and waste removal, good biocompatibility and an appropriate biodegradation rate. However, an increase in porosity coupled with pore size decreases (which is necessary for both bone ingrowth and nutrient supply) usually leads to the decrease of the biomechanical strength.

Several studies have focused on reinforcement of the porous scaffolds to compensate for this loss of biomechanical strength. Scaffolds have been synthesized utilizing bioceramic components, such as hydroxyapatite (HA) and tricalcium phosphate (TCP), mixed with biodegradable polymers, including poly(lactic-co-glycolic acid (PLGA) and polycaprolactone (PCL). However, bioceramics have poor biodegradability. Additionally, a disadvantage of the incorporation of ceramic powder is the poor interconnection with non-uniform pores within the closed porous structure. Further, bioceramics may cause phase separation into polymer blends upon exposure to organic solvents.

### Tissue Scaffold Fabrication Techniques

Studies have indicated that the mechanical properties of scaffolds also may be affected by the fabrication technique employed. The nanofiber self-assembly or electrospinning technique utilizes biomaterials with properties similar in scale and chemistry to that of the natural in vivo extracellular matrix (ECM). However, studies utilizing nanofibrous scaffolds have indicated that nanofiber meshes have limited cellular penetration depth due to the increased thickness of the nanofiber layers and the reduced pore size that is utilized for optimal mechanical properties. Textile technologies also have been utilized to provide non-woven polyglycolide scaffold structures. However, these technologies present difficulties with obtaining high porosity and regular pore size. The solvent casting & particulate leaching (SCPL) technique incorporates the steps of dissolving a polymer into a suitable organic solvent, then casting the solution into a mold filled with porogen particles, such as an inorganic salt (e.g., sodium chloride, crystals of saccharose, gelatin spheres or paraffin spheres). However, SCPL provides a limited thickness range, and uses organic solvents which must be fully removed to avoid any possible damage to the cells seeded on the scaffold. The gas foaming technique obviates the need for use of organic solvents and solid porogens. However, the excessive heat used during compression molding prohibits the incorporation of any temperature-labile material, such as proteins and growth factors, into the polymer matrix and the pores do not form an interconnected structure. The emulsification/freeze-drying technique also obviates the need for use of a solid porogen. However, this technique requires the use of solvents, results in pore sizes that are relatively small, and provides irregular porosity. Sintering techniques (i.e., methods for making objects from particulate material, by heating the material below its melting point until the particles adhere to each other), including those that are microsphere-based, have been utilized to synthesize structures with higher interconnectivity and mechanical strengths than those made via conventional methods. However, the low porosity exhibited by these sintered scaffolds may inhibit nutrient supply and cellular infiltration within the scaffolds. These techniques usually are limited by the insufficient mechanical strength of the scaffold due to the low polymer content caused by high porosity.

Additional studies have focused on the architecture of the scaffolds. Efforts utilizing multiphase structures, multilayer scaffolds with different pore sizes and porosity, and scaffolds of different composites, have failed to satisfy the requirements of bone replacement.

Porous, three-dimensional matrices comprising polymers for use in bone replacement have been prepared using various techniques. Coombes and Heckman (Biomaterials 1992 3:217-224) describe a process for preparing a microporous polymer matrix containing 50:50 poly (lactic acid-glycolic acid) (PLAGA):PLA and 25:75 PLA:PLAGA. The polymer is dissolved in poor solvent with heat and the gel is formed in a mold as the polymer cools. Removal of the solvent from the matrix creates a microporous structure. However, the actual pore size of this matrix (<2 μm) is inadequate for bone cell ingrowth, which requires a pore size falling within the range of 100-250 μm for cell ingrowth to occur. Further, the gel cast material undergoes a significant reduction in size (5-40%) due to the removal of the solvent, thus leading to problems in the production of specific shapes for clinical use. Since the amount of shrinkage varies from sample to sample, changing the mold size to compensate for the shrinkage may not result in a consistent implant size.

Particulate leaching methods, wherein void-forming particles are used to create pores in a polymer matrix have been described by Mikos et al., (Polymer 1994 35:1068-1077) and (Thomson et al., J. Biomater. Sci. Polymer Edn 1995 7:23-38. These methods produce highly porous, biodegradable polymer foams for use as cellular scaffolds during natural tissue replacement. The matrices are formed by dissolving PLA in a solvent followed by the addition of salt particles or gelatin microspheres. The composite is molded and the solvent is allowed to evaporate. The resulting disks then were heated slightly beyond the T_{g} for PLA (58°-60° C.) to ensure complete bonding of the PLA casing. Once cooled, the salt or gelatin spheres were leached out to provide a porous matrix. However, in both types of particulate leaching methods, the modulus of the matrix is significantly decreased by the high porosity. Thus, while these matrices might perform well as cellular scaffolds, in other applications such as bone replacement, their low compressive modulus may result in implant fracture and stress overloading of the newly formed bone. These problems may further lead to fractures in the surrounding bone and complete failure at the implantation site.

Silva et al. (Macrol. Biosci. 2004. 4:743-65), utilizing a sintering technique, developed a porous HA scaffold with an array of internal and porous aligned channels. However, the fabrication of such a scaffold is complex and the high temperatures required for sintering are not favorable for growth factor loading.

Light et al. (U.S. Pat. Nos. 5,595,621 and 5,514,151) described absorbable structures for ligament and tendon repair. The hydrogel-based spiral matrix of the prosthesis does not provide the appropriate mechanical properties required by bone tissue. Further, the architecture of the matrix organizes cell proliferation in an axial direction and inhibits cell infiltration and migration in a radial direction thus preventing the formation of the uniform three-dimensional cell growth desired in tissue engineering. The gapless architecture of the matrix imparts the drawbacks of cylindrical scaffolds.

Berman et al. (U.S. Pat. No. 6,017,366) described a resorbable interposition arthroplasty implant. The implant does not provide the appropriate mechanical properties required by bone tissue and does not mimic the architecture of the native extracellular matrix. Further, the implant does not allow for three-dimensional cell penetration or uniform media influx.

Sussman et al. (U.S. Pat. No. 5,266,476) described a fibrous matrix for in vitro cell cultivation. This fibrous matrix, composed of non-biodegradable polymers, fails to mimic the architecture of the native extracellular matrix. Further, the composition of the fibrous matrix may allow for complete cell invasion into the wall of the matrix, similar to disadvantages associated with cylindrical or tubular scaffolds.

Robinson et al. (Otolaryngol. Head and Neck Surg. 1995 112:707-713) disclose a sintering technique to produce a macroporous implant wherein bulk D,L-PLA is granulated, microsieved, and sintered slightly above the glass transition temperature of PLA (58°-60° C.). Sintering causes the adjacent PLA particles to bind at their contact point producing irregularly shaped pores ranging in size from 100-300 μm. While the implants were shown to be osteoconductive in vivo, degradation of PLA caused an unexpected giant cell reaction.

Laurencin et al. described a salt leaching/microsphere technique to induce pores into a 50:50 PLGA/HA matrix (Devin et al., J. Biomater. Sci. Polymer Edn 1996 7:661-669). In this method, an interconnected porous network is made by the imperfect packing of polymer microspheres. The porous matrix is composed of PLGA microspheres with particulate NaCl and HA. The particulate NaCl is used to widen the channels between the polymer microspheres. The hydroxyapatite is used to provide added support to the matrix and to allow for osteointegration. In this method, PLGA is dissolved in a solvent to create a highly viscous solution. A 1% solution of poly(vinyl alcohol) then is added to form a water/oil emulsion. Particulate NaCl and HA are added to the emulsion and the resulting composite mixture is molded, dried, and subjected to a salt leaching step in water. The resulting matrix is then vacuum dried, and stored in a desiccator until further use.

In vitro studies by Laurencin et al. showed osteoblast attachment and proliferation to the three-dimensional porous matrix produced by a salt leaching/microsphere method (Attawia et al., J. Biomed. Mater. Res. 1995 29:843-848; Attawia et al., Biochem. and Biophys. Res. Commun. 1995 213:639-644). However, during degradation in vitro, the mechanical strength of this matrix decreased to the lower limits of trabecular bone. Accordingly, in vivo implantation of this matrix may result in the mechanical failure of the implant or stress overloading of the newly regenerated osteoblasts.

The formation of functional tissues and biological structures in vitro requires extensive culturing to promote survival, growth and induction of functionality. In general, the basic requirements of cells must be maintained in culture, which include oxygen, pH, humidity, temperature, nutrients and osmotic pressure maintenance. Diffusion often is the sole means of nutrient and metabolite transport in standard cell culture. However, as a cell culture becomes larger and more complex, additional mechanisms must be employed to maintain the culture. The introduction of the proper factors or stimuli required to induce functionality must be satisfied. In many cases, simple maintenance culture is not sufficient. Growth factors, hormones, specific metabolites or nutrients, and chemical and physical stimuli may be required. Further, engineered tissue scaffolds generally lack an initial blood supply, thus making it difficult for any implanted cells to obtain sufficient oxygen and nutrients to survive and/or function properly.

A major disadvantage of many of the orthopaedic materials in current use is their lack of flexibility and inability to be custom fit to the implant site. Synthetic bone grafts generally are available in a generic form or shape which forces the surgeon to fit the surgical site around the implant. This may lead to increases in bone loss, trauma to the surrounding tissue and delayed healing time.

Accordingly, conventional tissue engineered scaffolds for bone have limited tissue ingrowth. This is due to the restrained nutrient supply imposed by intrinsic geometrical and structural characteristics of the scaffold. The nutrient requirements of the inner regenerated tissues of typical scaffolds exceed that provided supplied by the biomolecular transportation (via inward and outward diffusion) of these matrixes and scaffolds. Studies have shown that insufficient nutrient supply limits the adhesion of remaining cells on the surface of the scaffold. Further, the nutrient supply affects the migration of cells into the scaffold since cells seeded in inner areas of a scaffold tend to migrate towards the surface and higher nutrient concentrations.

The present invention provides a novel spiral in tubular scaffold structure that provides sufficient mechanical properties and supports a proper nutrient supply for cell growth and methods of use thereof. The present invention further provides for functionalized scaffolds that incorporate bioceramics, growth factors and cells.

### SUMMARY OF THE INVENTION

According to a first aspect, the present invention provides an integrated scaffold for bone tissue engineering, the integrated scaffold comprising (i) a tubular outer shell; and (ii) a spiral scaffold insert. According to one embodiment, the tubular outer shell comprises a polymer material that is a biodegradable material. According to another embodiment, the tubular outer shell is a polymer material that is a nonbiodegradable material. According to another embodiment, the tubular outer shell comprises a blend of at least one polymer material and at least one ceramic material. According to some such embodiments, the blend of at least one polymer material and at least one ceramic material is in the form of a microsphere. According to another embodiment, the tubular outer shell may be fabricated utilizing sintered microspheres.

According to another embodiment of the first aspect of the invention, the spiral scaffold insert comprises a polymer. According to some such embodiments, the polymer material is a poly(ester), or derivative thereof. According to some such embodiments, the polymer material is a poly(anhydride), or derivative thereof. According to some such embodiments, the polymer material is a poly(phosphazene), or derivative thereof. According to some such embodiments, the polymer material is a poly(lactide-co-glycolide) (PLGA), or derivative thereof. According to some embodiments, the spiral scaffold is a high porosity spiral scaffold. According to some such embodiments, the high porosity spiral scaffold is prepared by using a salt-leaching method. According to some embodiments, the spiral scaffold is a low porosity spiral scaffold. According to some such embodiments, the low porosity scaffold is prepared using a solvent evaporation method.

According to another embodiment of the first aspect of the invention, the spiral scaffold insert further comprises a nanofiber coating. According to some such embodiments, the nanofiber coating comprises at least one polymer. According to some such embodiments, the nanofiber coating comprises at least one active agent. According to some such embodiments, the nanofiber coating is applied with electrospinning. According to some such embodiments, a spiral scaffold insert further comprising nanofibers is assembled, the method of assembly comprising the steps of: (i) preparing polymer sheets using a solvent casting and/or salt leaching method; (ii) preparing polymer nanofibers using electrospinning; (iii) electrospinning the polymer nanofibers directly onto both sides of the polymer sheet and (iv) rolling the nanofiber-bearing polymer sheet into a spiral structure. In some such embodiments, the thickness of the nanofibers may be controlled by regulating electrospinning time.

According to some embodiments of the first aspect of the invention, the gap distance within the spiral scaffold insert is controlled. According to some such embodiments, the gap distance within the spiral scaffold insert is controlled by utilizing an inert template. According to some such embodiments, the template is a sheet of metal foil. According to some such embodiments, the template is a sheet of a deformable material that may be placed on a polymer sheet, rolled with the polymer sheet such that the template and the polymer sheet to form a spiral template within the resulting spiral scaffold, then removed from the spiral scaffold so as to leave behind the spiral gap. According to some such embodiments, the template is a sheet of copper foil. According to some such embodiments, the gap distances between the spiral layers of the spiral scaffold are uniform from one layer to the next. According to some such embodiments, the gap distances between the spiral layers of the spiral scaffold are different from one layer to the next. According to some such embodiments, the gap distances between the spiral layers of the spiral scaffold are between about 1 μm and 500 μm.

According to a second aspect, the present invention provides a method for fabricating an integrated scaffold for bone tissue engineering, the method comprising the steps of: (a) providing a tubular outer shell component; (b) providing a spiral scaffold component; (c) inserting the spiral scaffold component into the tubular outer shell component, wherein an interface is created between the outer edge of the spiral scaffold component and the inner edge of the tubular outer shell component; (d) applying a solvent to the interface, wherein each of the outer edge of the spiral scaffold component and the inner edge of the tubular outer shell component partially solubilizes and interacts to form a bond; and (e) removal of the solvent, thereby forming an integrated scaffold for bone repair or replacement. According to one embodiment, the solvent is DCM. According to another embodiment, the removal of the solvent is by evaporation.

According to a third aspect, the present invention provides a layer-by-layer method of coating a polymer surface with a ceramic. According to one embodiment, the present invention provides a method of coating a polymer surface with a ceramic, the method comprising the steps of: (a) providing a first polymer surface; (b) applying a second polymer onto the first polymer surface so as to form a second polymer surface; (c) applying a ceramic solution onto the second polymer surface such that the second polymer and the ceramic solution interact through electrostatic attraction to deposit a consistent bilayer onto the first polymer surface. According to some such embodiments the ceramic has a negative electrostatic charge in solution. According to another embodiment, the first polymer surface of step (a) further comprises at least one ceramic. According to another embodiment, the method further comprises depositing bilayers onto the polymer surface. According to some such embodiments, the number of bilayers is at least 2. According to some such embodiments, the ceramic is β-tricalcium phosphate (β-TCP). According to some such embodiments, the ceramic is hydroxyapatite (HAP).

According to a fourth aspect, the present invention provides a method of applying cell sheets onto a spiral scaffold, the method comprising steps: (a) providing a first polymer surface; (b) depositing a tannic acid solution onto the first polymer surface; (c) depositing a poly (N-isopropyl acrylamide) solution onto the tannic acid solution-bearing first polymer surface; (d) repeating steps (b)-(c) at least once; (e) washing the first polymer surface with a wash solution; (f) culturing cells on the first polymer surface of step (e) such that a cell sheet is formed; (g) applying the cell sheet of step (f) onto a sheet of nanofibrous porous polymer scaffold; (h) wrapping the nanofibrous porous polymer scaffold of step (g) to form a spiral scaffold. According to one embodiment, the cells of step (f) are osteoblasts.

### BRIEF DESCRIPTION OF THE DRAWINGS

The patent or application file contains at least one drawing executed in color. Copies of this patent or patent application publication with color drawings will be provided to the Office upon request and payment of the necessary fee.

The present invention will be further explained with reference to the accompanying drawings, wherein like structures are referred to by like numerals throughout the several views. The drawings shown are not necessarily to scale, with emphasis instead generally being placed upon illustrating the principles of the present invention.

FIG. 1A is an illustration of a spiral-in-tubular scaffold according to an embodiment of the present invention.

FIG. 1B is a top perspective view of the spiral scaffold insert of FIG. 1A.

FIG. 1C is a top view of the spiral scaffold insert of FIG. 1A.

FIG. 2 is a group of micrographs of a nanofibrous spiral scaffold insert according to an embodiment of the present invention.

FIG. 3 is a group of stereomicroscopic images of sintered tubular scaffolds, two of which are integrated with spiral scaffolds as inserts.

FIG. 4 is a pair of SEM photomicrographs of the interfaces of two integrated spiral-in-tubular bone scaffolds.

FIG. 5A is a bar graph of Young's modulus values obtained by mechanical testing of a cylinder scaffold, a tubular scaffold, an integrated porous scaffold, and an integrated fibrous scaffold.

FIG. 5B is a graph of yield strength values obtained by mechanical testing of a cylinder scaffold, a tubular scaffold, an integrated porous scaffold, and an integrated fibrous scaffold.

FIG. 6 is a graph of a tensile stress-strain curve.

FIG. 7A is a stereomicrograph of an integrated scaffold with a porous insert after pull-testing.

FIG. 7B is a stereomicrograph of an integrated scaffold with a fibrous insert after pull-testing.

FIG. 8 is a bar graph of changes in cell numbers plotted against time.

FIG. 9 is a bar graph of changes in alkaline phosphatase (ALP) activity during cell differentiation over a 21-day incubation period.

FIG. 10 is four groups of stereomicroscopic images of calcium deposits on four respective types of scaffolds stained with alizarin S red.

FIG. 11 is a bar graph of changes in calcium deposition upon four types of scaffolds over a 21-day incubation period.

FIG. 12 is a bar graph of cell numbers (as determined by the MTS assay) on eight scaffolds having different structures, over an 8-day incubation period.

FIG. 13 is a bar graph of ALP on each of the scaffolds of FIG. 12, over the 8 day incubation period.

FIG. 14 is a bar graph of calcium deposition on each of the scaffolds of FIG. 12.

FIG. 15 is three groups of SEM images of the surfaces of three respective scaffolds prior to cell seeding, and at stages of cell ingrowth.

FIG. 16 is a bar graph of the cell numbers on the scaffolds of FIG. 15, plotted against time observed during the 8-day incubation period as determined by the MTS assay.

FIG. 17 is a bar graph of changes in ALP activity during an 8-day culture of seeded human osteoblast cells.

FIG. 18 is a bar graph of the amount of calcium present on each of the scaffolds of FIG. 15 at the end of the 8-day culture.

FIG. 19A is a group of micrographs of fabricated spiral scaffolds.

FIG. 19B is a bar graph of calcium deposition on the spiral scaffold of FIG. 19A as estimated by alizarin red assay.

FIG. 20 is a bar graph of cell numbers estimated by MTS absorption (at 490 nm) for human osteoblast cells cultured on spiral scaffolds.

FIG. 21 is a bar graph of absorbance (405 nm) observed during an ALP assay of the scaffolds of FIG. 19A during a 28-day incubation of the seeded cells.

FIG. 22 is a bar graph of the calcium present on the scaffolds of FIG. 19A during a 28-day incubation of seeded cells.

FIG. 23 is a plot of protein released over time from five types of spiral scaffolds.

FIG. 24 is a plot of percentage release of Nerve Growth Factor from two types of scaffolds over time.

FIG. 25 is a pair of photomicrographs of cell sheets fabricated from temperature responsive substrates prepared by self-assembly.

FIG. 26 is a live-dead image of osteoblast cells on porous polymeric sheets.

FIG. 27 is a bar graph of MTS absorbance of cell sheets and a cell suspension during a 7-day culture.

FIG. 28 is a bar graph of ALP activity of cells in suspension and cell sheets during a 7-day culture.

### DETAILED DESCRIPTION

The present invention relates to tissue engineered scaffolds for the repair of bone defects and techniques for fabricating three-dimensional tissues for transplantation in human recipients.

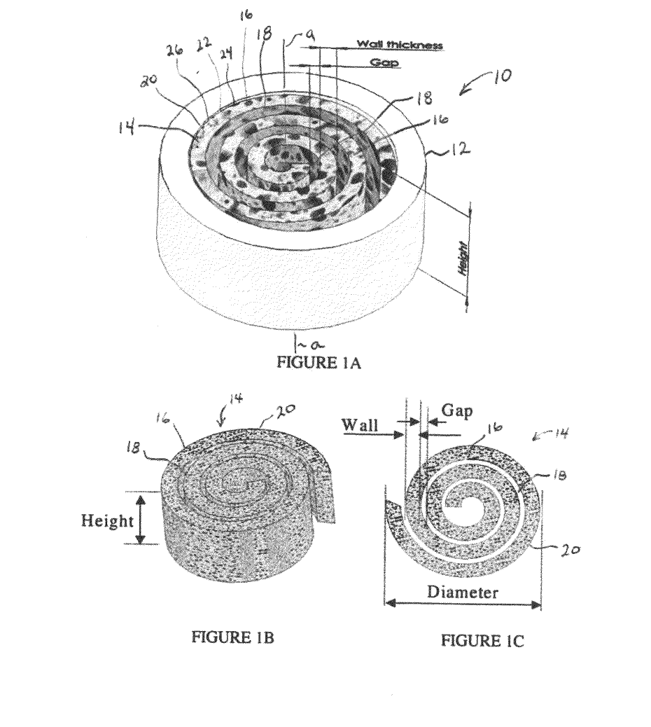

Referring to FIGS. 1A, 1B, and 1C, according to one aspect, the present invention provides an integrated scaffold 10 for bone tissue engineering, the integrated scaffold 10 comprising (i) a tubular outer shell; 12 and (ii) a spiral scaffold insert 14. The spiral scaffold insert 14 comprises a continuous series of spiral coils 16 (also referred to herein as “walls”) about an axis “a”. The coils 16 define a spiral gap 18. The spiral scaffold insert 14 may be conveniently formed from a single sheet 20 of a bioabsorbable polymer.

The term “integrated” as used herein refers to scaffolds that are organized or structured such that constituent units function synergistically.

The term “polymer” as used herein refers to a molecule composed of repeating structural units typically connected by covalent bonds. Polymers include, but are not limited to, cellulose, polysaccharides, polypeptides, polyproplylene, nylon, polystyrene, polyacrylonitrile, silicone, polyethylene, polyesters, polyanhydrides, polyphosphazene, poly(lactide-co-glycolide) (PLGA), poly(lactic acid) (PLA), poly(glycolic acid) (PGA), poly(lactic acid-glycolic acid) (PLAGA), poly(glaxanone), and poly(orthoesters).

According to one embodiment, the tubular outer shell 12 comprises a polymer material. In some such embodiments, the polymer material is a poly(ester), or derivative thereof. In some such embodiments, the polymer material is a poly(anhydride), or derivative thereof. In some such embodiments, the polymer material is a poly(phosphazene), or derivative thereof. In some such embodiments, the polymer material is PLGA, or a derivative thereof.

The term “biodegradable” as used herein refers to be capable of decaying through the action of living organisms or by enzymatic degradation.

According to another embodiment, the tubular outer shell 12 comprises a polymer material that is a biodegradable material. According to another embodiment, the tubular outer shell 12 is a polymer material that is a nonbiodegradable material.

According to yet another embodiment, the tubular outer shell 12 comprises a blend of at least one polymer material and at least one ceramic material. In some such embodiments, the ceramic material is hydroxyapatite (HA). In some such embodiments, the ceramic material is a calcium phosphate-based material. In some such embodiments, the ceramic material is tricalcium phosphate (TCP). In some such embodiments, the ceramic material is a composite comprising inorganic components. In some such embodiments, the ceramic material is a composite comprising inorganic and organic components. In some such embodiments, the ceramic material is based on silicate. In some such embodiments, the ceramic material is a bioactive glass. In some such embodiments, the tubular outer shell further comprises a glass-isomer.

According to some embodiments, the blend of at least one polymer material and at least one ceramic material is in the form of a microsphere. In some such embodiments, the ratio of the at least one polymer material to the at least one ceramic material is 1:1. In some such embodiments, the ratio of the at least one polymer material to the at least one ceramic material is 50:50% wt. In some such embodiments, the ratio of the at least one polymer material to the at least one ceramic material is 5:95% wt. In some such embodiments, the ratio of the at least one polymer material to the at least one ceramic material is 10:90% wt. In some such embodiments, the ratio of the at least one polymer material to the at least one ceramic material is 15:85% wt. In some such embodiments, the ratio of the at least one polymer material to the at least one ceramic material is 20:80% wt. In some such embodiments, the ratio of the at least one polymer material to the at least one ceramic material is 25:75% wt. In some such embodiments, the ratio of the at least one polymer material to the at least one ceramic material is 30:70% wt. In some such embodiments, the ratio of the at least one polymer material to the at least one ceramic material is 35:65% wt. In some such embodiments, the ratio of the at least one polymer material to the at least one ceramic material is 40:60% wt. In some such embodiments, the ratio of the at least one polymer material to the at least one ceramic material is 45:55% wt. In some such embodiments, the ratio of the at least one polymer material to the at least one ceramic material is 60:40% wt. In some such embodiments, the ratio of the at least one polymer material to the at least one ceramic material is 65:35% wt. In some such embodiments, the ratio of the at least one polymer material to the at least one ceramic material is 70:30% wt. In some such embodiments, the ratio of the at least one polymer material to the at least one ceramic material is 75:25% wt. In some such embodiments, the ratio of the at least one polymer material to the at least one ceramic material is 80:20% wt. In some such embodiments, the ratio of the at least one polymer material to the at least one ceramic material is 85:15% wt. In some such embodiments, the ratio of the at least one polymer material to the at least one ceramic material is 90:10% wt. In some such embodiments, the ratio of the at least one polymer material to the at least one ceramic material is 95:5% wt.

According to another embodiment, microspheres are fabricated using a solvent evaporation technique. First, a polymer material is dissolved in a solvent, such as, for example, but not limited to, methylene chloride. Second, the mixture is emulsified by pouring the mixture, with stirring, into an emulsifying agent solution, such as, but not limited to, 1% poly(vinyl alcohol). Third, upon evaporation of the solvent at room temperature (about 25° C.), the microspheres are isolated, washed with deionized water, dried, and sieved. In some such embodiments, ceramic particles may be loaded into the microspheres by adding the ceramic particles with the polymer, prior to the addition of the solvent. According to some such embodiments, individual microspheres may be of 500 μm to 800 μm in diameter. According to some such embodiments, individual microspheres may be of 550 μm to 750 μm in diameter. According to some such embodiments, individual microspheres may be of 610 μm to 710 μm in diameter. According to some such embodiments, individual microspheres may be of 100 μm to 206 μm in diameter

According to another embodiment, the tubular outer shell 12 may be fabricated utilizing microspheres. Microspheres are placed into a three-dimensional mold, then this assembly of microspheres is sintered (i.e., a sintered bond is formed between adjacent microspheres) to form a coherent mass by heating the microspheres without the application of pressure. This coherent mass may then be further processed by mechanical means to form tubular outer shells, such as tubular outer shell 12. For example, such tubular outer shells may be formed utilizing a drill press equipped with a heavy duty TiN-coated screw machine-length high speed steel drill bit. In some such embodiments, such tubular outer shells may also be formed utilizing a teflon (PTFE)-based mold with a stainless steel axis. In some such embodiments, the sintering temperature is from about 80-120° C. In some such embodiments, the sintering temperature is about 105° C. In some such embodiments, the sintering process is performed for about 1 hour. In some such embodiments, the sintering process is performed for about 2 hours. In some such embodiments, the sintering process is performed for about 3 hours. In some such embodiments, the mold is a stainless steel mold. According to some such embodiments, the mold is a teflon (PTFE)-based mold. According to some such embodiments, the tubular outer shell 12 may have a median pore diameter in the range of from about 50 μm to about 400 μm. According to some such embodiments, the tubular outer shell 12 may have a median pore diameter in the range of from about 100 μm to about 300 μm. According to some such embodiments, the tubular outer shell 12 may have a median pore diameter in the range of from about 150 μm to about 185 μm.

According to some embodiments, the spiral scaffold insert 14 comprises a polymer. In some such embodiments, the polymer material is a poly(ester), or derivative thereof. In some such embodiments, the polymer material is a poly(anhydride), or derivative thereof. In some such embodiments, the polymer material is a poly(phosphazene), or derivative thereof. In some such embodiments, the polymer material is PLGA.

The term “porosity” as used herein refers to the state or property of being porous. The term “porous” as used herein refers to admitting passage through pores, openings, holes, channels or interstices.

According to some such embodiments, the spiral scaffold insert 14 is a high porosity spiral scaffold. In some such embodiments, the high porosity spiral scaffold is prepared by using a salt-leaching method. This approach allows the preparation of porous structures with regular porosity, but with a limited thickness. First, the polymer is dissolved into a suitable organic solvent (for example, polylactic acid could be dissolved into dichloromethane), then the solution is cast into a mold filled with porogen particles. Such a porogen may be, but not limited to, an inorganic salt such as, but not limited to, sodium chloride, crystals of saccharose, gelatin spheres or paraffin spheres. The size of the porogen particles will affect the size of the scaffold pores, while the polymer to porogen ratio is directly correlated to the amount of porosity of the final structure. After the polymer solution has been cast the solvent is allowed to fully evaporate, then the composite structure in the mold is immersed in a bath of a liquid suitable for dissolving the porogen (for example, water in case of sodium chloride, saccharose and gelatin, or an aliphatic solvent like hexane for paraffin). Once the porogen has been fully dissolved a porous structure is obtained.

According to some such embodiments, the spiral scaffold insert 14 is a low porosity spiral scaffold. In some such embodiments, a low porosity spiral scaffold is prepared using a solvent evaporation method. This technique does not require the use of a solid porogen. First, a synthetic polymer is dissolved into a suitable solvent (for example, polylactic acid in dichloromethane) then water is added to the polymeric solution and the two liquids are mixed in order to obtain an emulsion. Before the two phases can separate, the emulsion is cast into a mold and quickly frozen. The frozen emulsion is subsequently freeze-dried to remove the dispersed water and the solvent, thus leaving a solidified, porous polymeric structure.

The phrase “fibrous spiral scaffold” or “nano-fibrous spiral scaffold” as used herein refers to a nanofiber-bearing polymer sheet rolled into a spiral structure. The phrase “porous spiral scaffold” as used herein refers to a spiral scaffold that may be prepared by solvent casting and/or salt leaching but without a nanofiber coating.

The term “electrospinning” as used herein refers to a process that utilizes an electrical charge to draw very fine (typically on the micro or nano scale) fibers from a liquid. Electrospinning shares characteristics of both electrospraying and conventional solution dry spinning of fibers. The process is non-invasive and does not require the use of coagulation chemistry or high temperatures to produce solid threads from solution.

According to some embodiments, the spiral scaffold insert 14 further comprises at least one active agent. According to some embodiments, the spiral scaffold insert 14 further comprises a nanofiber coating (not shown). According to some such embodiments, the nanofiber coating comprises at least one polymer. According to some such embodiments, the nanofiber coating comprises at least one active agent. According to some such embodiments, the nanofiber coating comprises at least one polymer and at least one active agent.

According to some such embodiments, the nanofiber coating is applied with electrospinning.

According to some such embodiments, the nanofiber coating is of a consistent thickness. According to some such embodiments, the consistent thickness varies in thickness across the surface to which the nanofiber coating has been applied less than 50% from one section to the next.

According to some such embodiments, a spiral scaffold insert, such as spiral scaffold insert 14, but further comprising nanofibers, is assembled, the method of assembly comprising the steps of: (i) preparing polymer sheets using a solvent casting and/or salt leaching method; (ii) preparing polymer nanofibers using electrospinning; (iii) electrospinning the polymer nanofibers directly onto both sides of the polymer sheet 20 and (iv) rolling the nanofiber-bearing polymer sheet 20 into a spiral structure. In some such embodiments, the polymer includes PCL. In some such embodiments, the thickness of the nanofibers may be controlled by regulating electrospinning time.

The term “gap distance” as used herein refers to the distance between two successive coils 16.

According to some embodiments, the gap distance within the spiral scaffold insert 14 is controlled. In some such embodiments, the gap distance within the spiral scaffold insert 14 is controlled by utilizing an inert template (not shown). In some such embodiments, the template is a sheet of metal foil. In some such embodiments, the template is a sheet of a deformable material that may be placed on a polymer sheet 20, rolled with the polymer sheet 20 such that the template and the polymer sheet 20 form a spiral template within the resulting spiral scaffold 14, (i.e., alternating coils of the polymer sheet 20 and the deformable material) then removed from the between the coils 16 so as to leave behind the spiral gap 18. In some such embodiments, the template is copper. In some such embodiments, the gap distances between the coils 16 of the spiral scaffold 14 are uniform from one coil 16 to the next. In some such embodiments, the gap distances between the coils 16 of the spiral scaffold 14 are different from one coil 16 to the next. In some such embodiments, the gap distances between the coils 16 of the spiral scaffold 14 are between about 1 μm and 500 μm. In some such embodiments, the gap distances between the coils 16 of the spiral scaffold 14 are between about 1 μm and 1000 μm. In some such embodiments, the gap distances between the coils 16 of the spiral scaffold 14 are between about 1 μm and 2000 μm. In some such embodiments, the gap distances between the coils 16 of the spiral scaffold 14 are between about 1 μm and 3000 μm. In some such embodiments, the gap distances between the coils 16 of the spiral scaffold 14 are between about 1 μm and 4000 μm. In some such embodiments, the gap distances between the coils 16 of the spiral scaffold 14 are between about 1 μm and 5000 μm. In some such embodiments, the gap distances between the coils 16 of the spiral scaffold 14 are between about 1 μm and 10000 μm.

According to another aspect, the present invention provides a method for fabricating an integrated scaffold, such as integrated scaffold 10, for bone tissue engineering, the method comprising steps: (a) providing a tubular outer shell component, such as tubular outer shell 12; (b) providing a spiral scaffold component, such as spiral scaffold insert 14; (c) inserting the spiral scaffold component 14 into the tubular outer shell component 12, wherein an interface 22 is created between the outer edge 22 of the spiral scaffold component and the inner edge 24 of the tubular outer shell component (see, e.g., FIG. 1A); (d) applying a solvent to the interface, wherein each the outer edge 24 of the spiral scaffold component and the inner edge 26 of the tubular outer shell component partially solubilize and interact to form a bond; and (e) removal of the solvent, thereby forming an integrated scaffold for bone repair or replacement.

According to one embodiment, the solvent is DCM. According to another embodiment, the removal of the solvent is by evaporation.

According to some embodiments, the tubular outer shell 12 and/or the spiral scaffold insert include an active agent. In some such embodiments, the active agent is a therapeutic agent. The terms “therapeutic agent” and “active agent” are used interchangeably herein to refer to a drug, compound, growth factor, nutrient, metabolite, hormone, enzyme, molecule, nucleic acid, protein, composition or other substance that provides a therapeutic effect. The term “therapeutic effect” as used herein refers to a consequence of treatment, the results of which are judged to be desirable and beneficial. A therapeutic effect may include, directly or indirectly, the arrest, reduction, or elimination of a disease manifestation. A therapeutic effect may also include, directly or indirectly, the arrest, reduction or elimination of the progression of a disease manifestation. A therapeutic effect may directly or indirectly kill the diseased cells, arrest the accumulation of diseased cells, or reduce the accumulation of diseased cells in a human subject with a disease, such as a pathological degeneration or congenital deformity of tissues.

In some such embodiments, the active agent is a drug. The term “drug” as used herein refers to a therapeutic agent or any substance, other than food, used in the prevention, diagnosis, alleviation, treatment, or cure of disease. A drug is: (a) any article recognized in the official United States Pharmacopeia, official Homeopathic Pharmacopeia of the United States, or official National Formulary, or any supplement to any of them; (b) articles intended for use in the diagnosis, cure, mitigation, treatment, or prevention of disease in man or other animals; (c) articles (other than food) intended to affect the structure or any function of the body of man or other animals, and d) articles intended for use as a component of any articles specified in (a), (b) or (c) above.

In some such embodiments, the active agent treats a disorder. The term “treat” or “treating” as used herein refers to accomplishing one or more of the following: (a) reducing the severity of a disorder; (b) limiting the development of symptoms characteristic of a disorder being treated; (c) limiting the worsening of symptoms characteristic of a disorder being treated; (d) limiting the recurrence of a disorder in patients that previously had the disorder; and (e) limiting recurrence of symptoms in patients that were previously symptomatic for the disorder. The term “disease” or “disorder” as used herein refers to an impairment of health or a condition of abnormal functioning. The term “syndrome” as used herein refers to a pattern of symptoms indicative of some disease or condition. The term “injury” as used herein refers to damage or harm to a structure or function of the body caused by an outside agent or force, which may be physical or chemical. The term “condition” as used herein refers to a variety of health states and is meant to include disorders or diseases caused by any underlying mechanism or disorder, injury, and the promotion of healthy tissues and organs. In some such embodiments, the disorder is a skeletal disorder. In some such embodiments, the skeletal disorder is a bone cyst, bone spur (osteophytes), bone tumor, craniosynostosis, fibrodysplasia ossificans progressiva, fibrous dysplasia, giant cell tumor of bone, hypophosphatasia, Klippel-Feil syndrome, metabolic bone disease, osteitis deformans, Paget's disease of bone, osteitis fibrosa cystica, osteitis fibrosa, Von Recklinghausens' disease of bone, osteitis pubis, condensing osteitis, osteitis condensans, osteitis condensans ilii, osteochondritis dissecans, osteochondroma, osteogenesis imperfecta, osteomalacia, osteomyelitis, osteopenia, osteopetrosis, osetoporosis, osteosarcoma, porotic hyperostosis, primary hyperparathyroidism, and renal osetodystrophy.

The therapeutic agent(s) may be provided in bits. The term “bits” as used herein refers to nano or microparticles (or in some instances larger) that may contain in whole or in part an active agent. The bits may contain the active agent(s) in a core surrounded by a coating. The active agent(s) also may be dispersed throughout the bit. The active agent(s) also may be adsorbed on at least one surface of the bit. The bits may be of any order release kinetics, including zero order release, first order release, second order release, delayed release, sustained release, immediate release, etc., and any combination thereof. The bits may include, in addition to the active agent(s), any of those materials routinely used in the art of pharmacy and medicine, including, but not limited to, erodible, nonerodible, biodegradable, or nonbiodegradable material or combinations thereof. The bits may be microcapsules that contain an active agent composition in a solution or in a semi-solid state. The bits may be of virtually any shape.

Both non-biodegradable and biodegradable polymeric materials may be used in the manufacture of bits for delivering the active agent(s). Such polymers may be natural or synthetic polymers. The polymer is selected based on the period of time over which release is desired. Bioadhesive polymers of particular interest include bioerodible hydrogels as described by Sawhney et al in Macromolecules (1993) 26, 581-587, the teachings of which are incorporated by reference herein. These include polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

The active agent(s) may be contained in controlled release systems. In order to prolong the effect of a drug, it often is desirable to slow the absorption of the drug. This may be accomplished by the use of a liquid suspension of crystalline or amorphous material with poor water solubility. The rate of absorption of the drug then depends upon its rate of dissolution which, in turn, may depend upon crystal size and crystalline form. The term “controlled release” is intended to refer to any drug-containing formulation in which the manner and profile of drug release from the formulation are controlled. This refers to immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that preferably, although not necessarily, results in substantially constant blood levels of a drug over an extended time period. Alternatively, delayed absorption of a drug form may be accomplished by dissolving or suspending the drug in an oil vehicle. The term “delayed release” is used herein in its conventional sense to refer to a drug formulation in which there is a time delay between administration of the formulation and the release of the drug there from. “Delayed release” may or may not involve gradual release of drug over an extended period of time, and thus may or may not be “sustained release.”

Use of a long-term sustained release implant may be particularly suitable for treatment of chronic conditions. The term “long-term” release, as used herein, means that the implant is constructed and arranged to deliver therapeutic levels of the active ingredient for at least 7 days, for at least 10 days, for at least 14 days, for at least about 21 days, for at least about 30 days, or for at least about 60 days. Long-term sustained release implants are well-known to those of ordinary skill in the art and include some of the release systems described above.

In some such embodiments, the active agent is a conventional nontoxic pharmaceutically-acceptable carrier, adjuvant, excipient, or vehicle. Examples of such carriers or excipients include, but are not limited to, calcium carbonate, calcium phosphate, various sugars, starches, cellulose derivatives, gelatin, and polymers such as polyethylene glycols. The term “pharmaceutically-acceptable carrier” as used herein refers to one or more compatible solid or liquid filler, diluents or encapsulating substances which are suitable for administration to a human or other vertebrate animal. The term “carrier” as used herein refers to an organic or inorganic ingredient, natural or synthetic, with which the active ingredient is combined to facilitate the application. The components of the pharmaceutical compositions also are capable of being commingled in a manner such that there is no interaction which would substantially impair the desired pharmaceutical efficiency. In some such embodiments, the active agent is bovine serum albumin (BSA).

In some such embodiments, the active agent is a growth factor. Such growth factors may include, but are not limited to, nerve growth factor (NGF), neurotrophins, brain-derived neurotrophic factor (BDNF), neurotrophin-3 (NT-3), neurotrophin-4 (NT-4), GFL, ciliary neurotrophic factor (CNTF), glia maturation factor (GMFB), neuregulin-1 (NRG1), neuregulin-2 (NRG2), neuregulin-3 (NRG3), neuregulin-4 (NRG4), epidermal growth factor (EGF), bone morphogenetic proteins (BMPs), which include BMP2, BMP3, BMP4, BMP5, BMP6, BMP7, BMP8a, BMP8b, BMP10, BMP15, vascular endothelial growth factor (VEGF), fibroblast growth factor (FGF), transforming growth factor beta (TGF-β), growth differentiation factors (GDF) which include, GDF1, GDF2, GDF3, GDF5, GDF6, GDF7, Myostatin/GDF8, GDF9, GDF10, GDF11, and GDF15.

According to another aspect, the present invention provides a layer-by-layer method of coating a polymer surface with a ceramic. According to one embodiment, the present invention provides a method of coating a polymer surface with a ceramic, the method comprising the steps of: (a) providing a first polymer surface; (b) applying a second polymer onto the first polymer surface so as to form a second polymer surface; (c) applying a ceramic solution onto the second polymer surface such that the second polymer and the ceramic solution interact through electrostatic attraction to deposit a consistent bilayer onto the first polymer surface. According to some such embodiments the ceramic has a negative electrostatic charge in solution. According to one embodiment, the first polymer surface of step (a) further comprises at least one ceramic.

According to another embodiment, the first polymer surface has a positive electrostatic charge. According to another embodiment, the first polymer surface has a negative electrostatic charge. According to another embodiment, the second polymer has a positive electrostatic charge. According to another embodiment, the second polymer has a negative electrostatic charge.

According to another embodiment, the method further comprises depositing multiple bilayers onto the polymer surface. According to some such embodiments, the number of bilayers is at least 2. According to some such embodiments, the number of bilayers is at least 3. According to some such embodiments, the number of bilayers is at least 4. According to some such embodiments, the number of bilayers is at least 5. According to some such embodiments, the number of bilayers is at least 10. According to some such embodiments, the number of bilayers is at least 25. According to some such embodiments, the number of bilayers is at least 50. According to some such embodiments, the number of bilayers is at least 100.

According to some such embodiments, the ceramic is β-tricalcium phosphate (β-TCP). According to some such embodiments, the ceramic is hydroxyapatite (HAP). According to some such embodiments, the ceramic solution comprises tannic acid. According to some such embodiments, the ceramic solution comprises β-TCP.

According to some such embodiments, the polymer surface is a spiral scaffold, such as spiral scaffold insert 14. According to some such embodiments, the polymer surface is a tubular outer shell, such as tubular outer shell 12. According to some such embodiments, the polymer surface is a fibrous scaffold. According to some such embodiments, the polymer surface is a porous scaffold. According to some such embodiments, the polymer surface is a cylindrical scaffold. According to some such embodiments, the polymer surface is a nanofiber. According to some such embodiments, the polymer surface is a spiral scaffold created by phase separation. According to some such embodiments, the polymer surface is an electrospun nanofiber. According to some such embodiments, the polymer surface is an electrospun nanofiber-coated phase-separated scaffold. According to some such embodiments, the polymer surface is a microsphere-sintered scaffold.

According to another aspect, the present invention provides a method of applying cell sheets onto a spiral scaffold, the method comprising the steps of: (a) providing a first polymer surface; (b) depositing a tannic acid solution onto the first polymer surface; (c) depositing a poly (N-isopropyl acrylamide) solution onto the tannic acid solution-bearing first polymer surface; (d) repeating steps (b)-(c) at least once; (e) washing the first polymer surface with a wash solution; (f) culturing cells on the first polymer surface of step (e) such that a cell sheet is formed; (g) applying the cell sheet of step (f) onto a sheet of nanofibrous porous polymer scaffold; (h) wrapping the nanofibrous porous polymer scaffold of step (g) to form a spiral scaffold.

According to one embodiment, the first polymer surface is that of a petri dish. According to some such embodiments, the first polymer surface of step (a) is coated with PEI/PLL. According to another embodiment, step (d) is repeated five times. According to another embodiment, the wash solution is sterile PBS. According to another embodiment, the wash solution is DMEM. According to another embodiment, the cells of step (f) are osteoblasts. According to some embodiments, the nanofibrous porous polymer scaffold of step (g) is a PCL nanofibrous scaffold. According to some embodiments, the cell sheet of step (g) further comprises extracellular matrix proteins.

Cell culture methods useful in the present invention are described generally in numerous well-known textbooks and manuals pertaining to cell culture. Tissue culture supplies and reagents useful in the present invention are well-known and are available from commercial vendors such as Gibco/BRL, Nalgene-Nunc International, Sigma Chemical Co., and ICN Biomedicals.

General methods in molecular genetics and genetic engineering useful in the present invention are described in numerous well-known textbooks and manuals pertaining to molecular genetics and genetic engineering. Reagents, cloning vectors, and kits for genetic manipulation that are useful in the present inventions are well-known and are available from commercial vendors such as BioRad, Stratagene, Invitrogen, ClonTech and Sigma-Aldrich Co.

Where a value of ranges is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and” and “the” include plural references unless the context clearly dictates otherwise. All technical and scientific terms used herein have the same meaning.

Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Fabrication of Spiral-in-Tubular Scaffolds

### Example 1.1

### Low Porosity Poly(ε-Caprolactone) (PCL) Scaffolds

Poly(ε-caprolactone) (PCL) sheets (50-100 μm in thickness) were fabricated using a solvent evaporation method. Briefly, PCL in dichloromethane (DCM) (33% w/v) was spread on the surface of a glass Petri dish and the DCM was evaporated leaving a PCL sheet. The PCL sheet was then rolled along with a piece of copper sheet that acted as a mold to form a low-porosity PCL spiral scaffold. After incubation in an oven (45° C. for 30 minutes), the scaffold was immediately transferred to ice cold water for at least 24 hours to immobilize its shape. The copper mold was then removed from the low porosity scaffold prior to further experimentation.

### Example 1.2

### High Porosity PCL Spiral Scaffolds

High porosity PCL spiral scaffolds were prepared using a salt-leaching method. Briefly, sodium chloride (NaCl) crystals (150-300 μm in size) and NaCl particles (200 μm in size) were added to a PCL/DCM solution (33% w/v) in a 1:1 (w/w) ratio. The mixture was spread onto a glass Petri dish and the spiral scaffolds were formed as described above. The scaffolds were then submerged into deionized water to remove the salt. The resulting spiral scaffolds proved to be highly porous.

### Example 1.3

### PCL Electrospun Nanofiber and PCL Fiber-Coated-Highly Porous PCL Spiral Structured Scaffolds

PCL spiral scaffolds having PCL nanofiber coatings were fabricated using an electrospinning technique. Briefly, PCL (200 mg) was dissolved in hexafluoroisopropanol (2 ml). The polymer solution was delivered at a constant flow rate to a metal capillary connected to a high voltage source. Charged polymer nanofibers were deposited on both sides of a previously fabricated PCL sheet. The sheet was then formed into a spiral scaffold using the method described above. The spiral scaffolds formed in this Example have lengths of about 5 mm, inner diameters of about 1 mm, outer diameters of about 10 mm, gap widths of about 15 μm and wall thicknesses of about 400 μm.

### Example 1.4

### Fabrication of Poly(Lactide-co-Glycolide) (PLGA) Sintered Microsphere Matrices

Biodegradable polymeric microspheres were fabricated from PLGA copolymer (85:15 lactide:glycolide) using an oil-in-water emulsion technique. Briefly, PLGA was dissolved in methylene chloride at 20% (w/v). The solution was slowly poured into a 1% (w/v) polyvinyl alcohol solution stirring at 250 rpm. The solvent was allowed to evaporate overnight at 25° C. under constant stirring. The microspheres were collected by vacuum filtration and washed with distilled water. Microspheres (106-212 μm diameter) were placed into three-dimensional molds and sintered (80° C. for 3 hours) to form cylindrical or tubular PLGA scaffolds.

### Example 1.5

### Spiral and Tubular Scaffold Integration

Spiral scaffolds, such as those described in Example 1.3, were inserted into the tubular scaffolds and the interface between the inner surface of the tubular scaffold and the outer surface of the respective spiral scaffold was sealed with DCM. Briefly, a small amount (3 μl) of DCM was added to the interface to partially solubilize the polymers of the respective surfaces and attach them to each other. A solidified bond between the tubular scaffold and the spiral scaffold formed following solvent evaporation. The scaffolds were dried in a vacuum to remove excess solvent prior to in vitro testing.

### Example 2

### Characterization of Integrated Spiral-in-Tubular Scaffolds

The integrated spiral-in-tubular scaffolds of Example 1.5 were characterized for surface morphology, porosity, mechanical properties and in vitro cell attachment and proliferation.

### Example 2.1

### Surface Morphology

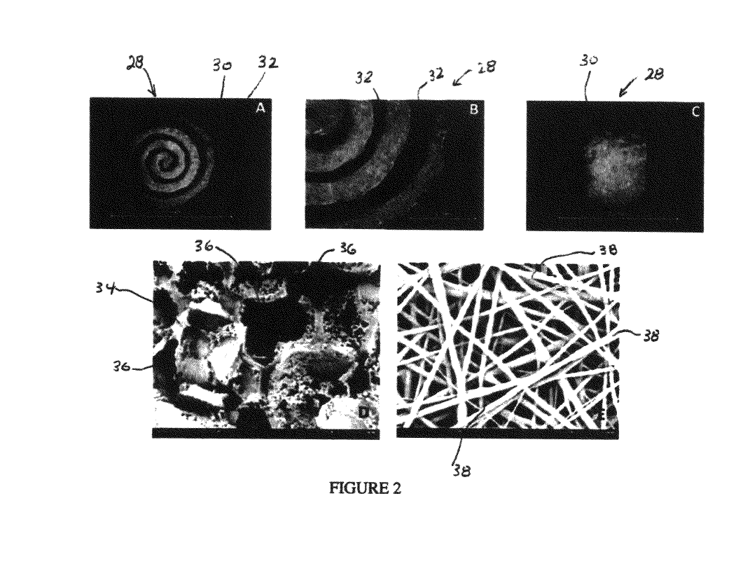

Nanofibrous PCL spiral scaffolds were observed using scanning electron microscopy (SEM). FIG. 2 shows photomicrographs of such scaffolds, including: (A) a top view of the spiral architecture of a spiral scaffold 28 showing the coil 30 and gap 32 architecture; (B) a detailed view of the uniform coil-gap structure and open architecture of spiral scaffold 28, (C) a side view of the spiral scaffold 28; (D) a scanning electron micrograph showing the porous surface 34, in which pores 36 are exemplary pores, prior to nanofiber loading; and (E) the surface architecture of the scaffold 28 coated with electrospun nanofibers 38. For SEM analysis, scaffold 28 was gold-coated for 25 seconds and examined for pore shape, pore interconnectivity, morphology, and structure. Qualitative analysis (see Abramoff, M. D., Magelhaes, P. J., Ram, S. J. “Image Processing with ImageJ”. Biophotonics International, volume 11, issue 7, pp. 36-42, 2004, which is incorporated by reference herein in its entirety) of the SEM images of the scaffolds allows for an estimate of the pore size. Results of the analysis also indicated that nanofibers were uniformly distributed over the surface of the scaffolds.

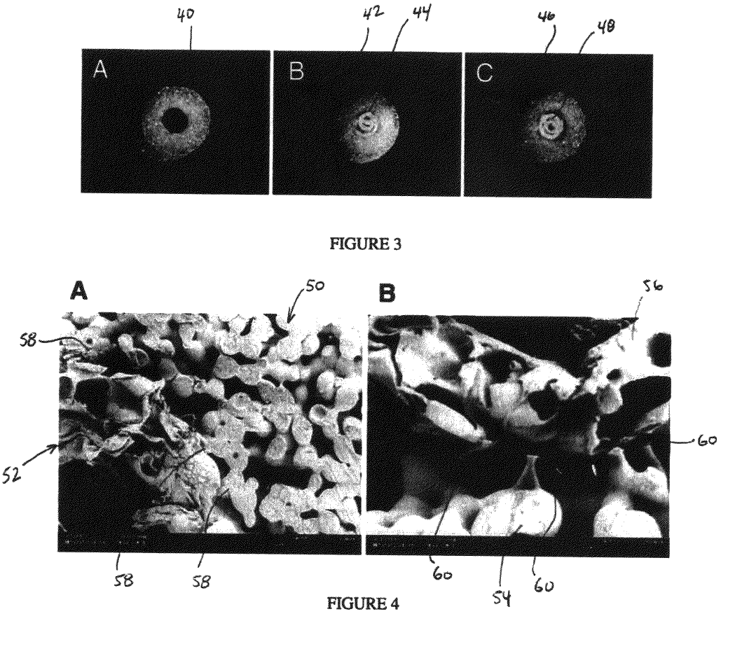

The fabrication of spiral-in-tubular scaffolds was confirmed with stereomicroscopy. FIG. 3 shows stereomicroscopic images of (A) a PLGA tubular scaffold 40, (B) a PLGA tubular scaffold 42 with a spiral PCL porous insert 44, and (C) a PLGA tubular scaffold 46 with a spiral PCL fibrous insert 48. These images also show variations in the gap distances and wall thicknesses of the porous and fibrous inserts.

The uniformity of integration of the components of the integrated scaffolds was further confirmed with SEM. FIG. 4 shows SEM photomicrographs of (A) a tubular scaffold 50 integrated with a porous spiral insert 52 and (B) a tubular scaffold 54 integrated with nanofiber-coated porous spiral insert 56. Examples of adhesions 58 between tubular scaffold 50 and porous spiral insert 52 and adhesions 60 between tubular scaffold 54 and nanofiber-coated porous spiral insert 56 can be observed in the respective microphotographs A and B.

### Example 2.2

### Porosity

Porosity analysis was performed utilizing (i) stereomicroscope imaging of the cross-section of the scaffolds; and (ii) a gravimetric method.

Table 1 shows that increases in porosity were obtained upon inclusion of a spiral insert coupled the inner surface of a tubular scaffold having an inner diameter of 2 mm.

TABLE 1

| | Porosity Measured by | | | Porosity Measured by | | |
|---|---|---|---|---|---|---|
| | Gravimetry method | | | Image Analysis | | |
| ID | Without | With spiral | With fibrous | Without | With spiral | With fibrous |
| (mm) | insert (%) | insert (%) | insert (%) | insert (%) | insert (%) | insert (%) |
| 2 | 42.33 ± 1.22 | 48.98 ± 1.51 | 48.01 ± 1.65 | 30.52 ± 3.49 | 46.05 ± 3.16 | 43.74 ± 2.26 |
| 0 | 42.02 ± 0.34 | — | — | 34.59 ± 1.59 | — | — |

### Example 2.3

### Mechanical Testing

Tubular scaffolds, cylindrical scaffolds, integrated porous scaffolds (i.e., non-fibrous), and integrated fibrous scaffolds were separately studied to determine whether the integration of the two components (i.e., the tubular scaffold and the spiral insert scaffold) affected the mechanical strength of the outer rigid tubular scaffolds. The mechanical properties of compressive strength and compressive modulus of the various scaffolds were determined using an Instron 1127 mechanical testing machine (Instron, Norwood, Mass.) according to the well-known methods for determining such mechanical properties.

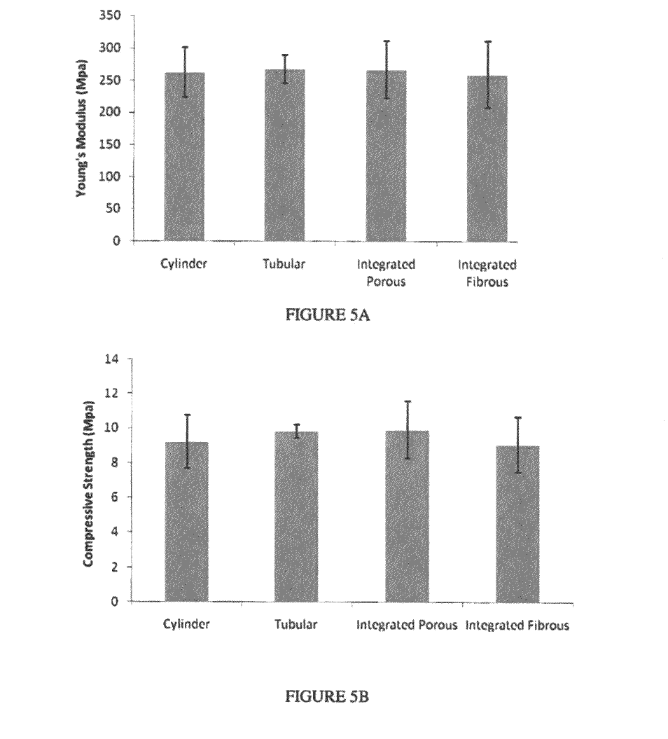

FIGS. 5A and 5B are bar graphs showing the Young's modulus and compressive strength values, respectively, obtained for the scaffolds tested. The error bars indicate 5 standard deviations. The use of the asterisks (i.e., “*”) on some bars signifies that the values shown are significantly greater (p<0.05) than the values for the cylindrical samples. Mechanical testing of PLGA sintered cylindrical and tubular scaffolds showed that there is no significant difference of Young's modulus and compressive strength between tubular scaffolds (ID=2 mm) and cylindrical scaffolds. Compressive testing was performed on tubular shells with integrated PCL spiral scaffold inserts to study the effect of integration on the mechanical strength of the scaffolds. The result showed no significant decrease of the Young's modulus and compressive strength of the scaffolds after integration.

### Example 2.4

### Pull-Out Testing

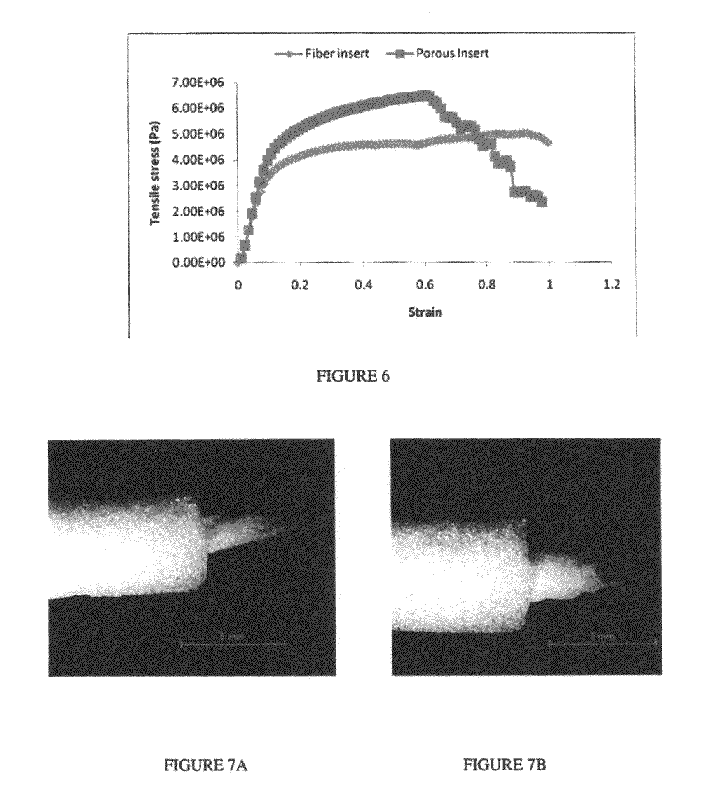

Pull-out testing was performed via a typical load-extension tensile test utilizing a RSA III Dynamic Mechanical Analyzer (TA Instruments, New Castle, Del.). This allowed analysis of the bonding strength between the outer surface of the spiral insert and the inner surface of the tubular scaffold of the integrated scaffolds. FIG. 6 is a graph of the load-strain curve recorded for the measurement of tensile strength or debonding strength, which shows that the porous insert had a higher bonding strength than the fibrous insert. Stereomicroscopy was utilized to inspect the integrity of the integrated scaffolds after the pull-out test. FIG. 7A is a stereomicrograph of the integrated scaffold having a porous insert and FIG. 7B is a stereomicrograph of the integrated scaffold having a fibrous insert. Both scaffolds maintained structural integrity (i.e., the outer surfaces of the spiral inserts did not debond from the inner surface of the tubular scaffolds).

### Example 3

### Cell Attachment, Proliferation Phenotypic Expression and Mineralized Matrix Deposition on the Integrated Spiral in Tubular Structured Scaffolds

### Example 3.1

### Cell Proliferation

Human osteoblast cells (hFOB 1.19, ATCC) were adopted as model cells for the preliminary evaluation of cellular responses on the nanofibrous scaffolds. Scaffolds were sterilized in an 70% ethanol bath (one hour), irradiated with UV light (30 minutes) in PBS, then washed 3 times with PBS. The scaffolds then were equilibrated in fresh medium (30 minutes) to facilitate the cell adhesion. The experiment was conducted in 24-well plates with one scaffold in each well. A concentrated human osteoblast cell suspension (40 μl) was dropped evenly from the top side of the scaffolds for maximum cellular attachment of each scaffold to provide a final cell seeding density of 5×10⁴ cells per scaffold. Additional medium (1 ml) was added to each well after 2 hours of incubation to provide for maintenance of the cell culture on the scaffolds. For cylinder scaffolds, most of the cells were located at the surface.

Cells were cultured in the a medium containing a 1:1 mixture of Ham's F12 medium (GIBCO) and Dulbeccco's Modified Eagle Medium-Low Glucose (DMEM-LG; Sigma) supplemented with antibiotic solution (1% penicillin-streptomycin; Sigma) and 10% fetal bovine serum (FBS; Sigma), 1% β-glycerophosphate (Sigma) and maintained in a humidified atmosphere of 5% CO2 at 37° C. The media were changed every 2 days, and the cultures were maintained for 21 days. At days 4, 8, 14 and 21, scaffolds were removed and characterized for cell proliferation, differentiation, mineralized matrix synthesis, and morphological analysis.

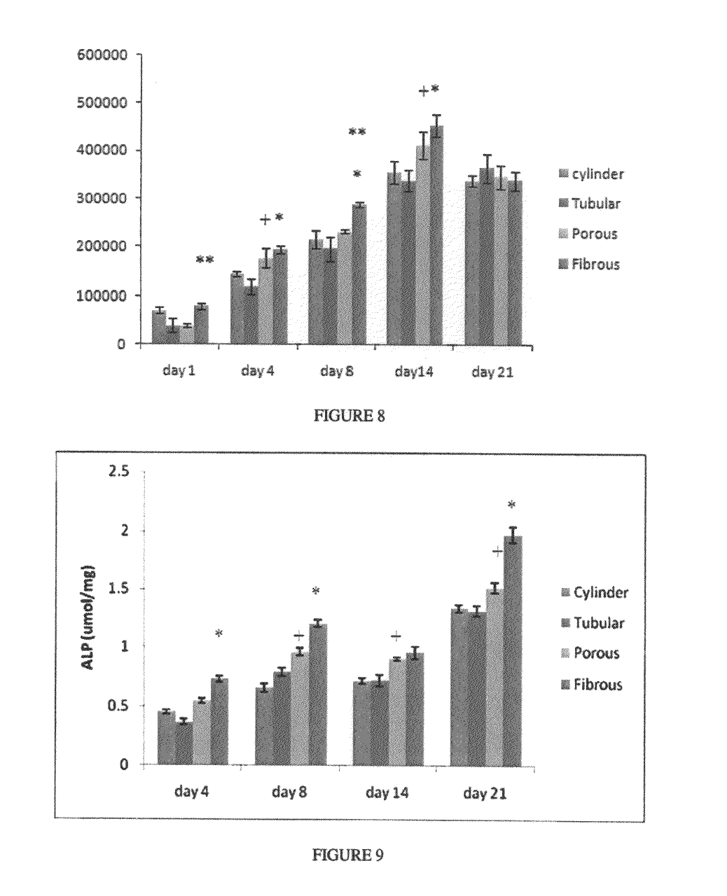

The cell proliferation at day 4, day 8, day 14 and day 21 were analyzed by the 3-(4,5-dimethylthiazol-2-yl)-5-(3-carboxymethoxyphenyl)-2-(4-sulfophenyl)-2H-tetrazolium MTS assay. After incubation, cell numbers were determined by using the MTS assay kit (Promega, Madison, Wis.) according to the manufacturer's protocol. FIG. 8 is a bar chart showing cell numbers (as determined by the MTS assay) plotted against time. The error bars indicate 3 standard deviations. The “*” indicates that the numbers of cells on the fibrous scaffolds at days 4, 8 and 14 were significantly greater (p<0.05) than those on the cylindrical and tubular scaffolds. The “+” indicates that the numbers of cells on the porous scaffolds at days 4 and 14 were significantly greater (p<0.05) than those on the cylindrical scaffolds. The “**” indicates that the cell numbers on the integrated fibrous scaffolds at days 1 and 8 were significantly higher (p<0.05) than those on the porous scaffolds. For all scaffolds, the numbers of cells peak at day 14, then decrease by day 21.

### Example 3.2

### Alkaline Phosphatase (ALP) Activity

The early development of the osteoblast-like phenotype was evaluated by measuring ALP activity. Briefly, scaffolds were removed from the wells, washed (3 times) with PBS, then freeze/thawed (3 times) and treated with 1% Triton-5 mM MgCl₂ (1 ml) to extract the intracellular alkaline phosphatase. The resultant solution containing ALP was analyzed using 1 mg/ml P-NPP (Sigma) in 1× diethanolamine substrate buffer (Pierce). A mixture containing sample (20 μl) and reagent (100 μl) was incubated at 37° C. for 30 minutes until a bright yellow color appeared. At the end of the incubation time, the absorbance at 405 nm was measured with a microplate reader (BioTek). The results for ALP activity were normalized by the total protein amount in each well (determined by using Quickstart Bradford Protein assay kit (Bio-Rad)). Samples (5 μl) (same as solution used for ALP assay) were incubated with reagent (250 μl) for 5 minutes, then the absorbance at 595 nm measured with a micro plate reader (BioTek). The protein amount was determined through a standard curve that was established using a list of known standard bovine serum albumin (BSA) solutions.

FIG. 9 is a bar chart of the alkaline phosphatase (ALP) activity of cells over a 21 day time course. The error bars indicate 3 standard deviations. At early time points, (i.e., from day 4 to day 14), cells on all scaffolds exhibited low level of ALP expression. The cells on the integrated scaffolds exhibited higher ALP activity than those on the cylindrical scaffolds and tubular scaffolds throughout the time course. For example, ALP activities were significantly higher on integrated porous scaffolds were significantly higher (p<0.05) than ALP activities on cylindrical or tubular scaffolds on days 8, 14 and 21, as designated by a plus (i.e., “+”) sign. Further, the cells on the integrated fibrous scaffolds exhibited significantly higher ALP activity than those cells on the integrated porous scaffolds on days 4, 8 and 21, as designated by an asterisk (i.e., “*”).

### Example 3.3

### Matrix Mineralization

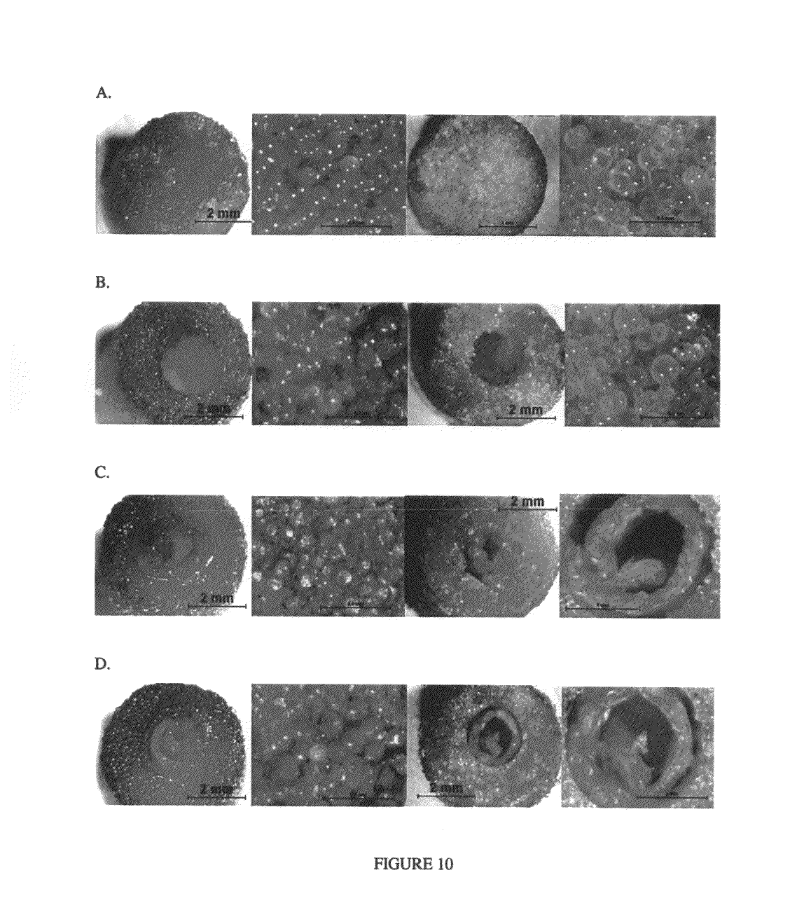

The deposition of calcium (Ca) on the scaffolds was analyzed using alizarin red assay. This assay allows to qualitatively determine deposited calcium through images and also quantitatively measure the extent of deposition of calcium, indicating matrix mineralization. FIG. 10 shows four groups of four color stereomicroscopic images each of calcium deposits (red) on four respective groups of scaffolds (Group A: cylindrical scaffolds; Group B: tubular scaffolds; Group C: integrated porous scaffolds; and Group D integrated fibrous scaffolds) stained with alizarin S red after 21 days. Each Group A-D of images shows respectively from left to right, (i) an end surface view; (ii) an enlarged end surface view; (iii) lateral cross-sectional view; and (iv) enlarged lateral cross-sectional view of each of the respective scaffolds. Analysis demonstrates that mineral matrix formed within the tubular scaffolds (Group B), integrated porous scaffolds (Group C) and integrated fibrous scaffolds (Group D); low amounts of deposited CA were observed within the cylinder scaffolds (FIG. 10A).

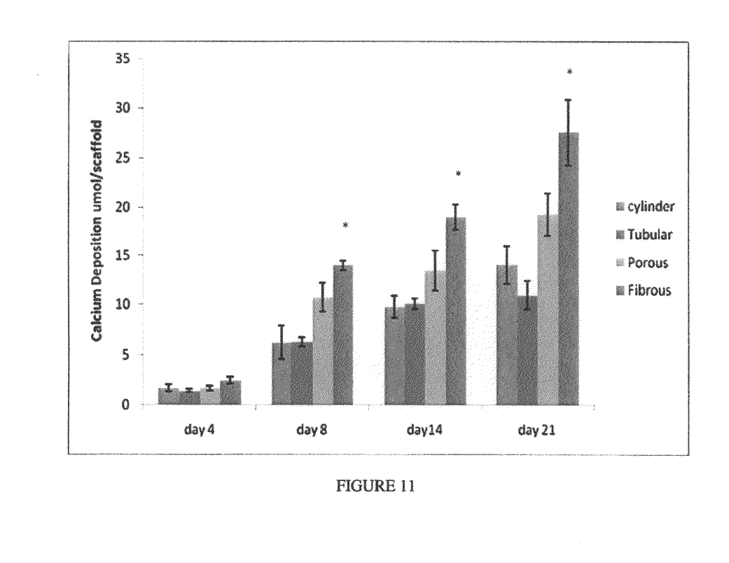

FIG. 11 is a bar chart showing the calcium deposition (in μmol Ca/scaffold) upon the cylinder scaffolds, tubular scaffolds, integrated porous scaffolds and integrated fibrous scaffolds, the images of which are shown in FIG. 10. Error bars indicate 3 standard deviations. The presence of an asterisk (i.e., “*”) indicates that the amount of calcium deposited on the integrated fibrous scaffold was significantly higher (p<0.05) than the amounts deposited on the other three types of scaffolds on the day in question. Mineralized matrix synthesis at days 4, 8, 14 and 21 were quantitatively analyzed with the alizarin red staining method for calcium deposition. Briefly, the scaffolds were fixed with 4% formaldehyde at 4° C. for 30 minutes, then stained with 2% alizarin red (Sigma) solution for 10 minutes. To quantify the amount of calcium on the scaffold, the red matrix precipitate was solubilized in 10% cetylpyridinium chloride (Sigma), and the optical density of the solution was read at 562 nm with a micro-plate reader (BioTek). The amount of calcium deposition was expressed as molar equivalent of CaCl₂ per scaffold. The integrated scaffolds exhibited higher levels of calcium deposition than either the tubular scaffolds or cylinder scaffolds; the integrated fibrous scaffold exhibited the highest levels of calcium deposition. These results suggest the addition of the fibrous spiral inserts increases cell differentiation and cell phenotype development upon tubular scaffolds.

### Example 4

### Characterization of the Structure of the Inner Nanofibrous Spiral Scaffolds

### Example 4.1

### Effect of Gap Distance

Eight groups of nanofibrous three-dimensional scaffolds having different gap distances and wall thicknesses (Table 2) were fabricated according to the procedures described in Examples 1.1-1.5, then utilized for characterization studies.

TABLE 2

| | | Wall | Gap | |
|---|---|---|---|---|
| | | Thickness | Distance | |
| Group | Feature | (mm) | (mm) | Fiber Coating |
| 1 | 0.2 mm (Tight) | 0.2 | 0.05 | Not applied |
| 2 | 0.2 mm | 0.2 | 0.05 | Electrospun fibers |
| | (Fiber/Tight) | | | (2 minutes) |
| 3 | 0.4 mm (Tight) | 0.4 | 0.05 | Not applied |
| 4 | 0.2 mm | 0.4 | 0.05 | Electrospun fibers |
| | (Fiber/Tight) | | | (2 minutes) |
| 5 | 0.2 mm (Gap) | 0.2 | 0.2 | Not applied |
| 6 | 0.2 mm (Fiber/Gap) | 0.2 | 0.2 | Electrospun fibers |
| | | | | (2 minutes) |
| 7 | 0.4 mm (Gap) | 0.4 | 0.2 | Not applied |
| 8 | 0.4 mm (Fiber/Gap) | 0.4 | 0.2 | Electrospun fibers |
| | | | | (2 minutes) |

### Example 4.1.1

### Cell Proliferation on Scaffolds With Varying Gaps and Wall Thicknesses

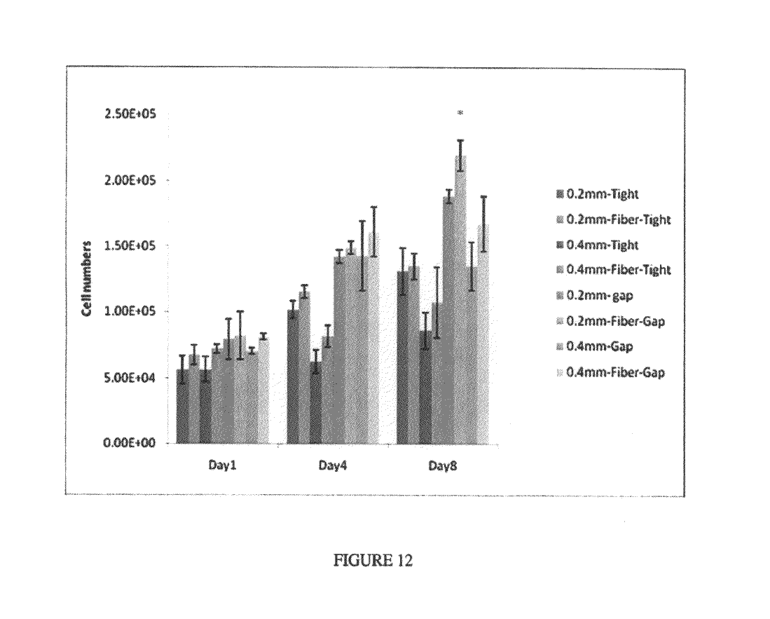

Human osteoblast cells (ATCC) were utilized as model cells for the evaluation of cell proliferation on the eight groups of scaffolds (Table 2). Human osteoblast cells were seeded onto the scaffolds at a density of 1.5×10⁵ cells per scaffold. After 1, 4 and 8 days of incubation, cell numbers were determined using the MTS assay kit. FIG. 12 is a bar chart of the cell numbers (as determined by the MTS assay) on each scaffold over the 8 day incubation period. The error bars indicate 3 standard deviations. The numbers of cells on the scaffolds with gaps between the spiral layers (“open structure spiral scaffolds”) were higher than those of scaffolds without gaps between the spiral layers (“tight spiral scaffolds”). Additionally, the number of cells on the scaffolds with thinner wall thickness (0.2 mm) was higher than those of scaffolds with thicker wall thickness (0.4 mm). Further, after 8 days of incubation, the Group 6 scaffolds (0.2 mm gap, fibrous insert) had the highest number of cells present as indicated by the presence of an asterisk (i.e., “*”). These results demonstrate that altering the geometry (gap distance, wall thickness) of the scaffold may influence cell proliferation on the scaffolds.

### Example 4.1.2

### Cell Differentiation of Scaffolds With Varying Gaps and Wall Thicknesses

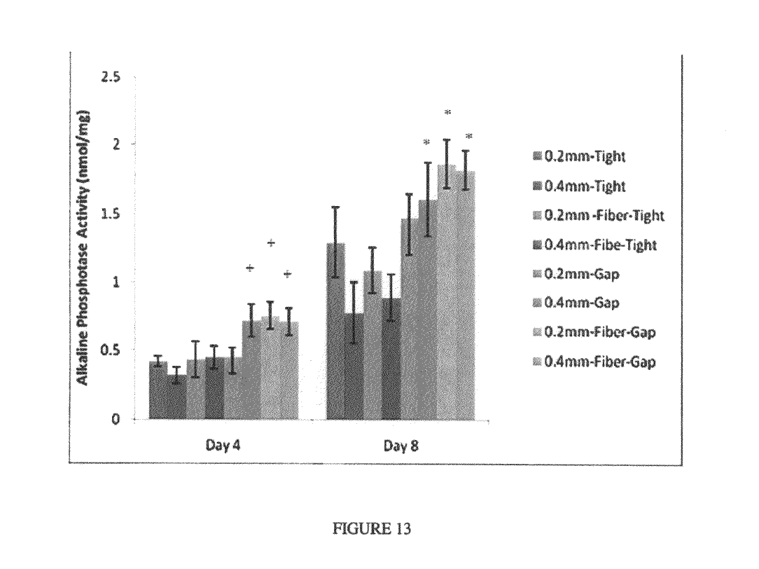

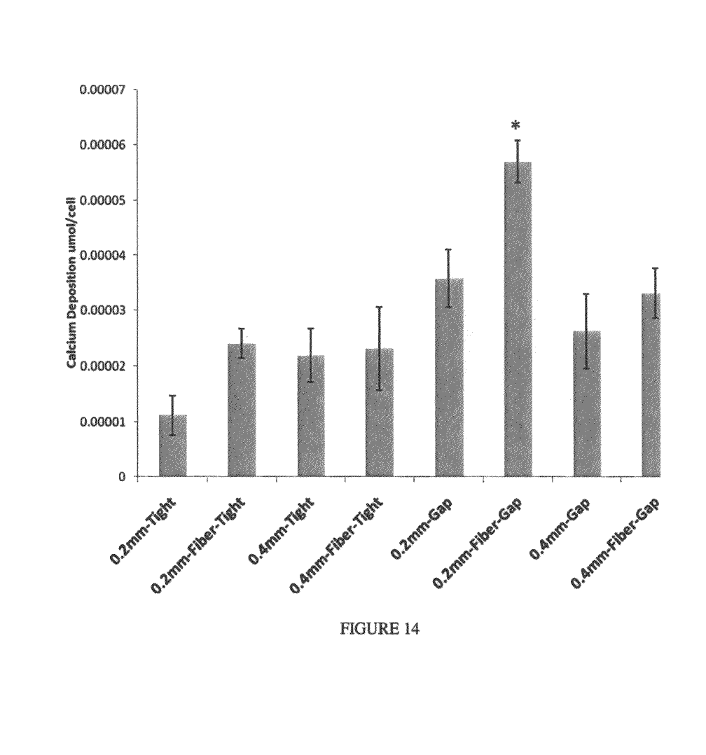

The level of cell differentiation of the cells on the scaffolds (Table 2) as illustrated by the expression of ALP and by extracellular matrix mineralization was studied. Human osteoblast cells were seeded onto the scaffolds at a density of 1.5×10⁵ cells per scaffold, then incubated for 8 days. Osteoblastic differentiation of the seeded cells was analyzed utilizing an ALP assay (as described in Example 3.2). Matrix mineralization was analyzed using an alizarin red assay for calcium deposition (as described in Example 3.3). FIG. 13 is a bar chart of the ALP activity (nmol/mg) on each scaffold over the 8 day incubation period. Generally, those spiral scaffolds with gaps exhibited higher ALP activity than those spiral scaffolds without gaps. The presence of a plus (i.e., “+”) sign on day 4 and asterisk (i.e., “*”) on day 8 indicates that the ALP activity on a spiral scaffold with gaps was significantly greater (p<0.05) than the ALP activity on a spiral scaffold without gaps. Further, those spiral scaffolds with gaps and fibrous inserts exhibited the highest ALP activity. FIG. 14 shows a graph of the calcium deposition (μmol/cell) on each group of scaffold after the 8-day incubation. The fibrous spiral scaffold with gaps and a thinner wall thickness exhibited the highest amount of calcium deposition, as indicated by the presence of an asterisk (i.e., “*”). These results demonstrate that altering the geometry (gap distance, wall thickness) of the scaffold may influence cell differentiation on the scaffolds.

### Example 4.2

### Effect of Fiber Thickness on Cell Attachment and Infiltration

Three-dimensional spiral scaffolds were fabricated as described herein to study the influences of fiber thickness upon the scaffolds. The PCL sheets were made using the solvent evaporation method, as described in Example 1.1. Briefly, PCL in dichloromethane (DCM) (33% w/v) was spread onto the surface of a glass petri dish and the DCM evaporated under reduced pressure to form a dry PCL thin layer. PCL in hexafluoroisopropanol (HFIP) (10%) (Oakwood Products, Inc., West Columbia, S.C.) then was electrospun into nanofibers with a constant flow rate (Q=0.08 ml/minute, KD Scientific syringe pump) to a metal capillary connected to a high-voltage power supply (Gamma High Voltage Research ES-30P, Ormond Beach, Fla.), then deposited onto the surface of the PCL porous sheet. The sheet was rolled along with a copper mold to form a spiral structure. After incubation in an oven (45° C. for 10 minutes), the scaffold was immediately transferred to ice cold water for at least 24 hours to immobilize the shape. The copper mold was removed prior to further experimentation. Different electrospinning times were utilized to fabricate three groups of nanofibrous scaffolds (Fiber 0 (0 second electrospin time); Fiber 1 (120 second electrospin time); and Fiber 2 (300 second electrospin time)).

### Example 4.2.1

### Cell Attachment, Infiltration, Matrix Deposition Into the Scaffold

Human osteoblast cells were seeded onto Fiber 0 scaffolds, Fiber 1 scaffolds, and Fiber 2 scaffolds at a density of 1.5×10⁵ cells per scaffold, incubated for 8 days, then analyzed for cell proliferation and cell infiltration utilizing scanning electron microscopy (SEM) and the MTS assay. The phenotypic expression of these seeded cells were analyzed utilizing ALP and alizarin red assays as described in Example 3.2 and Example 3.3, respectively.

### Example 4.2.2

### Cellular Infiltration Into Nanofibrous Scaffolds

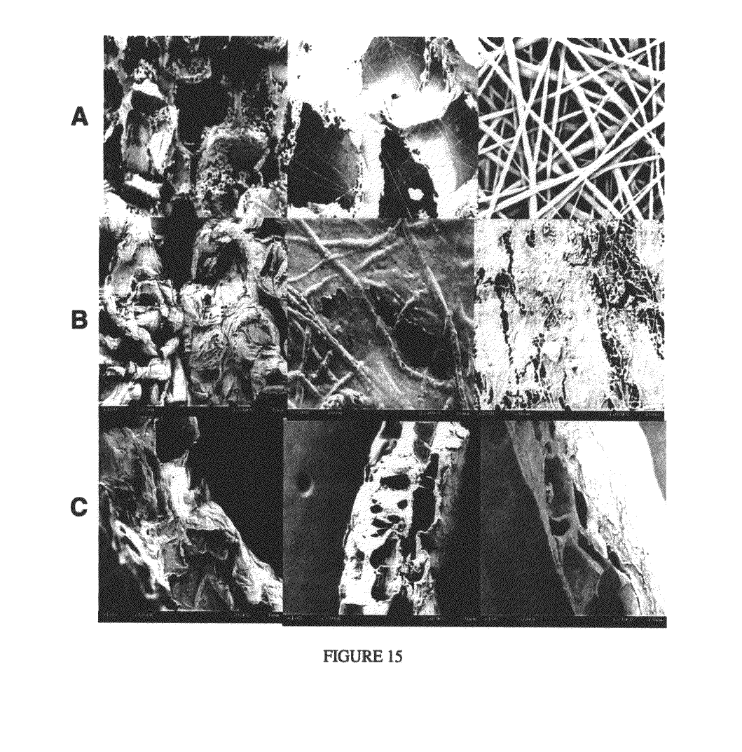

FIG. 15 presents three groups of micrographs (Groups A, B and C) of the surface of the Fiber 0 scaffold (leftmost in each of Groups A, B and C), Fiber 1 scaffold (middle of each of Groups A, B and C)and Fiber 2 scaffold (rightmost of each of Group A, B and C) before and after seeding with human osteoblast cells. In Group A the Fiber 0 scaffold has a porous structure and a pore size within the range of 150-300 μm; the Fiber 1 scaffold has randomly oriented fibers deposited on the porous surface and a pore size within the range of 50-100 μm; the Fiber 2 scaffold has randomly oriented fibers deposited on the porous structure and a pore size within the range of 5-10 μm. After seeding of each scaffold with cells, and a subsequent 8 day incubation period, cellular growth could be seen on the surfaces of Fiber 0, Fiber 1 and Fiber 2, as shown in the micrographs of Group B. The cross-sections of the Fiber 0 scaffold, Fiber 1 scaffold and Fiber 2 scaffold showed cellular penetration into each scaffold, as shown in the micrographs of Group C. Further, the Fiber 1 scaffold, shown in the center micrograph of Group C, demonstrated greater cellular penetration than the Fiber 2 scaffold, as shown in the right-most micrograph of Group C. These results suggest the presence of nanofibrous scaffolds allows for cellular infiltration into scaffolds.

### Example 4.2.3

### Cell Proliferation on Nanofiber-Coated Spiral Scaffolds

Cell proliferation upon the Fiber 0 scaffold, the Fiber 1 scaffold and Fiber 2 scaffold after cell seeding was studied.

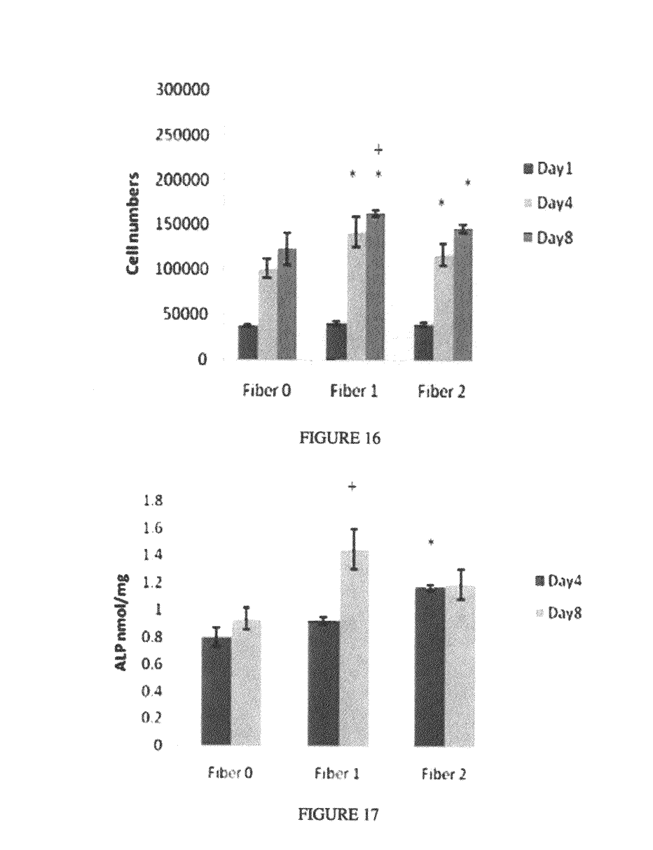

FIG. 16 is a bar chart showing the cell numbers of the Fiber 0 scaffold, the Fiber 1 scaffold and Fiber 2 scaffold observed during the 8-day incubation period as determined by the MTS assay. The “*” indicates a statistically significant higher (p<0.05) cell number at days 4 and 8 at the Fiber 1 and Fiber 2 scaffolds than at the Fiber 0 scaffold. The “+” indicates a statistically significant higher (p<0.05) cell number at day 8 at the Fiber 1 scaffold than at the Fiber 0 or Fiber 2 scaffold at day 8. Error bars indicate 3 standard deviations. The nanofibrous scaffolds (Fiber 1 and Fiber 2 scaffolds) had higher numbers of cells present throughout the culture period than the scaffold without nanofibers present (Fiber 0 scaffold). Further, the Fiber 1 scaffold (with a pore size range of 50-100 μm) had the highest number of cells present after 8 days. These results suggest the presence of nanofibrous scaffolds allows for cell proliferation upon the scaffolds.

### Example 4.2.4

### Cellular Differentiation on Nanofibrous Spiral Scaffolds

The early development of the osteoblast-like phenotype was evaluated by measuring ALP activity. FIG. 17 is a bar chart of the amount of ALP activity (ALP nmol/mg) during an 8-day culture of seeded human osteoblast cells. The “*” indicates a statistically significant higher (p<0.05) ALP activity at day 4 at the Fiber 2 scaffold than at the Fiber 0 scaffold or Fiber 1 scaffold. The “+” indicates a statistically significant higher (p<0.05) ALP activity at day 8 at the Fiber 1 scaffold than at the Fiber 0 or Fiber 2 scaffold. Error bars indicate 3 standard deviations. After 8 days, the fibrous scaffolds (Fiber 1 scaffold and Fiber 2 scaffold) exhibited the highest ALP activity.

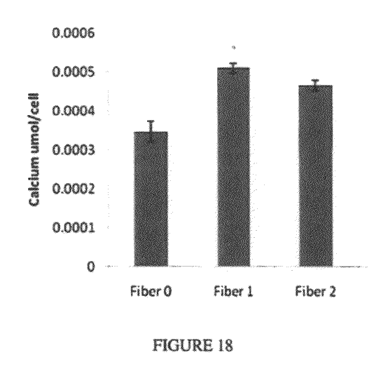

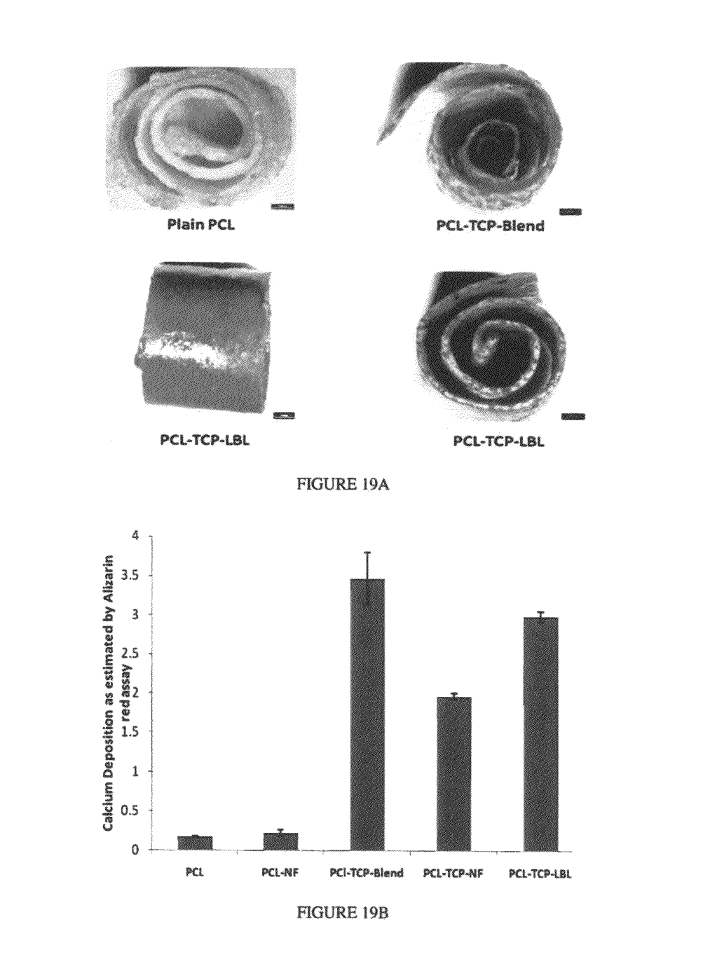

Calcium matrix deposition upon the spiral scaffolds was studied utilizing an alizarin red assay. FIG. 18 is a bar chart of the amount of calcium (μmol/cell) present on each of the Fiber 0 scaffold, the Fiber 1 scaffold and Fiber 2 scaffold. The “*” indicates a statistically significant higher (p<0.05) calcium deposition amount than at Fiber 0 and Fiber 2 scaffolds. Error bars indicate 3 standard deviations. Analysis of calcium deposition upon each scaffold indicated the fibrous scaffolds (Fiber 1 scaffold and Fiber 2 scaffold) exhibited higher calcium deposition than the nonfibrous scaffold (Fiber 0 scaffold). These results suggest that the presence of nanofibrous scaffolds allows for cellular differentiation of cells upon the scaffolds.

### Example 5

### Functionalization of Inner Nanofibrous Spiral Scaffolds

### Example 5.1

### Incorporation of β-Tricalcium Phosphate (β-TCP) onto Nanofibrous Spiral Scaffolds Utilizing Layer-by-Layer Deposition Technique

Layer-by-layer deposition technique was used to create nanoscale coatings. An electrostatic interaction between the ceramic and the surface was achieved by deposition of positively charged chitosan on the surface, alternated by a negatively charged solution of tannic acid-TCP solution.

Five bilayers of positively and negatively charged polymers were deposited and compared against scaffolds fabricated as ceramic blends (PCL-TCP) or by electrospinning of the ceramic onto the scaffold. Scaffolds fabricated by the layer-by-layer technique demonstrated improved cell proliferation, differentiation and matrix mineralization as compared to the ceramic blend scaffold and the electrospun scaffold.

In order to estimate the TCP deposition on the scaffold, an alizarin red staining assay was utilized to image and quantify the uniformity of deposition as well as the amount of calcium phosphate present in the scaffolds. From the images of the stained scaffolds (FIG. 19A) and bar chart of calcium quantification of the scaffolds (FIG. 19B) it is evident that physiologically relevant quantities of calcium was deposited on the scaffolds. Further, the images of the alizarin red stained scaffolds (FIG. 19A) showed uniform deposition as compared to blends of polymer and TCP based electrospun scaffolds and bulk films.

### Example 5.2

### Cell Attachment and Proliferation on Spiral Structured Scaffolds

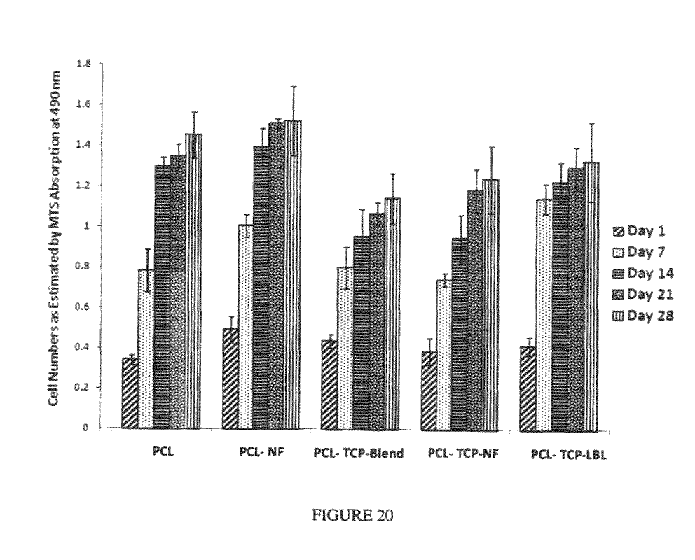

Human osteoblast cells (ATCC) were utilized as model cells for the preliminary evaluation of cellular response on the nanofibrous scaffolds, TCP-containing scaffolds produced by electrospinning, blended films and layer by layer technique. Human osteoblast cells were seeded at a density of 1.5×10⁵ cells per scaffold. Cell numbers were determined using an MTS assay kit after 1 day, 7 days, 14 days, 21 days and 28 days of incubation. FIG. 20 is a bar chart of cell numbers estimated by MTS absorption at 490 nm for human osteoblast cells cultured on a PCL spiral scaffold, a PCL nanofibrous scaffold (PCL-NF), a PCL-TCP blend scaffold, a PCL-TCP-nanofibrous scaffold (PCL-TCP-NF) and a PCL-TCP scaffold coated with LbL (PCL-TCP-LbL) during the 28 day incubation. At least 3 scaffolds from each group were analyzed. The “*” indicates a statistically significant higher (p<0.05) cell number on plain nanofibrous scaffolds at day 14 and day 21 as compared to TCP containing nanofibrous spiral scaffolds; “**” indicates a statistically significant higher (p<0.05) cell number on PCL-TCP-LBL scaffolds as compared to PCL-TCP-blends and PCL-TCP-NF on day 7 and day 14. Error bars indicate 3 standard deviations. The number of cells on the nanofiber containing spiral structured bone grafts was significantly higher as compared to that on the spiral structured scaffolds without nanofibers. It also was observed that cell attachment and proliferation on TCP containing scaffolds was lower than on scaffolds with nanofibers but without TCP. This difference in cell numbers has been attributed to the difference in surface topography and nano-indentations on the surfaces. However, cell proliferation on PCL-TCP-LBL scaffolds was higher as compared to PCL-TCP Blend as well as PCL-TCP-NF as of days 14 and 21. Without being limited by theory, this may be because the layer-by-layer development of scaffolds may lead to a smoother nanotopography owing to the incorporation of other polymers (tannic acid and chitosan), thereby improving cell proliferation on these scaffolds.

### Example 5.3

### Alkaline Phosphatase (ALP) Activity

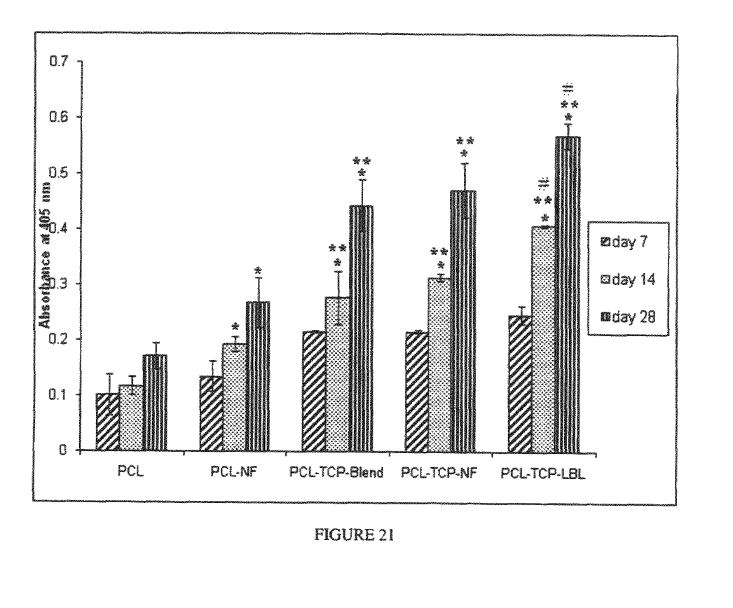

The early development of the osteoblast-like phenotype was evaluated by measuring ALP activity. FIG. 21 is a bar chart of absorbance (405 nm) demonstrated by a PCL scaffold, a PCL-NF scaffold, a PCL-TCP-blend scaffold, a PCL-TCP-NF scaffold and a PCL-TCP-LBL scaffold during a 28 day incubation of the seeded cells. At least 3 scaffolds from each group were analyzed. Error bars denote standard deviation. “*” indicates a statistically significant higher (p<0.05) amount of expressed ALP on fibrous scaffolds than porous scaffolds at day 14 and day 21; “**’ indicates a statistically significant higher (p<0.05) amount of expressed ALP on TCP containing scaffolds as compared to scaffolds without TCP on day 14 and day 28. “#” indicates a statistically significant increase in ALP activity of PCL-TCP-LBL scaffolds as compared to PCL-TCP blend scaffolds on day 14 and day 28. Quantitative intracellular ALP measurements on scaffolds containing osteoblast cells, in vitro, on nanofiber-containing PCL spiral scaffolds was higher as compared to PCL scaffolds without nanofibers. Also, the inclusion of TCP on the scaffolds showed a significant difference in terms of ALP production from cells. It was evident that nanofibrous coating on the spiral scaffolds had an impact on ALP activity over days 14 and 28; no significant difference was noticed in scaffolds for day 7. It also can be noted that TCP-containing scaffolds had enhanced ALP activity over days 14 and 28. Without being limited by theory, this may be due to signaling effect and better communication between cells and substrates (coated with TCP) owing to the better osteoconductivity. Also, the PCL-LBL-TCP scaffolds, showed increased ALP activity as compared to PCL-TCP-Blend and PCL-TCP-NF over days 14 and 28. Without being limited by theory, this effect could arise from more uniform TCP deposition on the surface as well as better communication between TCP and cells.

### Example 5.4

### Matrix Mineralization onto TCP-Containing Scaffolds

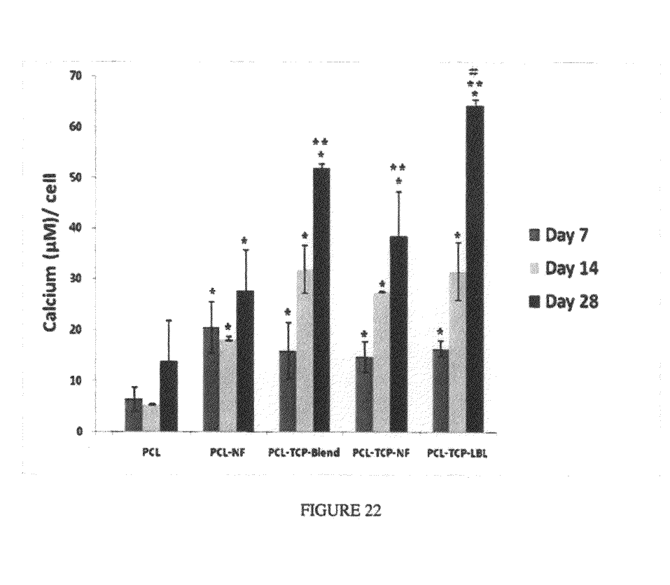

The deposition of calcium on the scaffolds was analyzed using alizarin red assay. This assay allows qualitative determination of deposited calcium through images and also quantitative measurements of the extent of deposition of calcium, indicating matrix mineralization. FIG. 22 is a bar chart of the amount of calcium (μM/cell) present on a TCP scaffold, a PCL scaffold, a PCL-NF scaffold, a PCL-TCP-blend scaffold, a PCL-TCP-NF scaffold and a PCL-TCP-LBL scaffold during a 28 day incubation of seeded cells. The “*” indicates a statistically significant (p<0.05) increase in calcium deposition amount on nanofiber-containing scaffolds as compared to plain spiral scaffolds and tissue cultured polystyrene as of days 7, 14 and 28; “**” indicates a statistically significant (p<0.05) increase in calcium deposition by cells on scaffolds containing TCP as compared to plain nanofibrous PCL scaffolds for day 28. “#” indicates a statistically significant (p<0.05) increase for PCL-TCP-LBL scaffolds over PCL-TCP blends and PCL-TCP nanofibrous scaffolds for day 28. The error bars indicate 3 standard deviations. Analysis demonstrated that the nanofibrous scaffolds produced significantly higher levels of calcium as compared to plain PCL spiral scaffolds. Also, the inclusion of TCP to the surface of the scaffolds improved the matrix mineralization properties of the scaffolds as compared to non functionalized spiral scaffolds. It also can be observed that the inclusion of TCP to the surface of the scaffolds (PCL-TCP nanofibers and PCL-TCP-LBL) had enhanced levels of matrix mineralization as compared to PCL-TCP blended scaffolds and PCL nanofibrous scaffolds. Without being limited by theory, this could be due to the nanofiber coating on the surface, which may block cellular interactions with the TCP loaded inside the scaffolds (in the form of a blend), indirectly affecting matrix mineralization.

### Example 6

### Functionalization of Inner Nanofibrous Spiral Scaffolds by Incorporation of Proteins Through Controlled Delivery

### Example 6.1

### Drug Release from Scaffolds

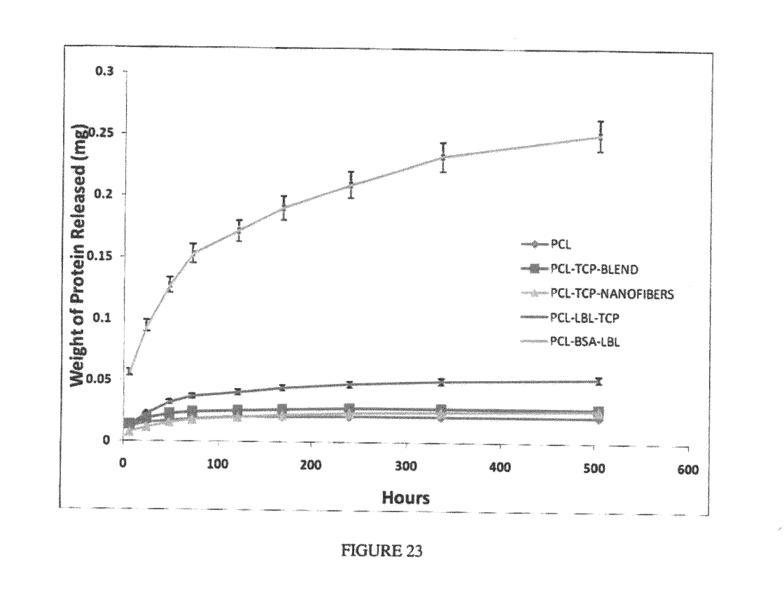

The release of a model drug, bovine serum albumin (BSA), was analyzed to evaluate controlled release from scaffolds. The fabrication steps were defined above and five types of scaffolds, similar to the ones used for the cell studies in Examples 4.1-4.2.4, were evaluated. The BSA was loaded into the nanofibers, similar as in Example 6.2. Multilayers were prepared by Layer-by-layer (LBL) technique on top of the nanofibers and the release was evaluated (for sample PCL-BSA-LBL). For sample PCL-LBL-TCP, the multilayers were prepared first and the BSA was loaded similar to the other scaffolds tested in this study. The term “burst” as used herein refers to a release of a high percentage of a drug over a short period of time (generally 24 hours). FIG. 23 is a plot of protein released (mg) against time (hours) from a PCL scaffold, a PCL-TCP-blend scaffold, a PCL-TCP-NF scaffold, a PCL-LBL-TCP scaffold and a PCL-BSA-LBL scaffold. The plain PCL scaffolds and the PCL-TCP blended scaffolds, had an increased burst as compared to other scaffolds. On studying 28 day release profiles from the scaffolds, it was observed that LBL prepared scaffolds had a very similar release profile to other samples that were tested. Both of these scaffolds had increased amounts of BSA loaded in the scaffolds as compared to other techniques, as well as release significantly higher amount of BSA over 28 days; however, the amount of release was significantly increased in the LBL samples due to increased loading of the BSA in the system.

### Example 6.2

### Controlled Release of Nerve Growth Factor From Nanofibrous Coating

Nanofibers of a bovine serum albumin (BSA) and polycaprolactone (PCL) blend were fabricated and analyzed. PCL-BSA solution was prepared by dissolving 100 mg of PCL and 50 mg of BSA in 1 ml hexafluroisopropanol. Then 100 μl of 10 μg/ml solution of nerve growth factor (NGF; adopted as a model protein for these release studies) in PBS was added and was stirred to dissolve the NGF in the PCL-BSA blends. This solution was electrospun at 12 kV at a flow rate of 10 μL/min on a grounded aluminum foil. Controlled release of NGF was evaluated by placing 40 mg of fibers in 1 mL of RPMI media followed by incubation at 37° C. Release samples were collected at the predetermined time points (1, 4, 7, 14, 21 and 28 days) and were quantified using a NGF ELISA kit. In order to determine the bioactivity of released NGF, the release samples were introduced into PC12 cells cultured on 24 well plates. PCL-NGF nanofibers were used as controls. The cells were allowed to differentiate for five days and were imaged using an inverted microscope at 25× in order to determine neurite length. An average of 200 cells was counted per well from 5 distinct frames for determining the average neurite lengths and standard deviations. The Student t-test was used for statistical analysis and a p<0.05 was considered statistically significant.

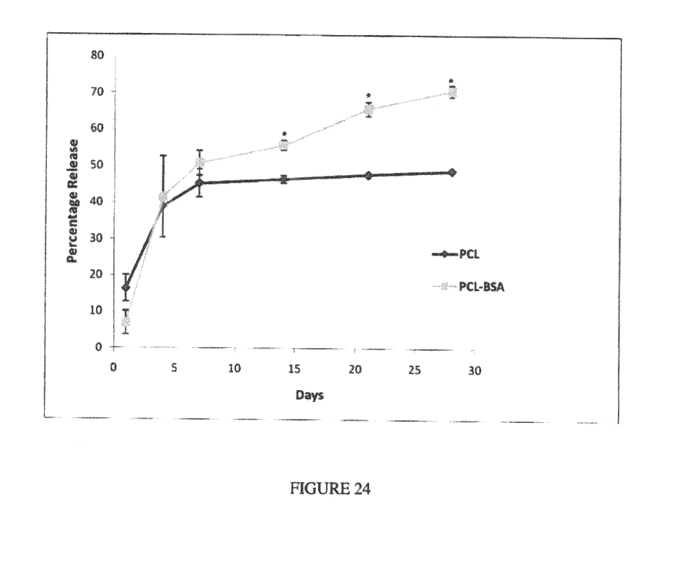

FIG. 24 is a plot of percentage release of NGF against time (days). The “*” indicates statistically significant difference of NGF release from PCL-BSA nanofiber scaffolds as compared to PCL nanofiber scaffold. From the data, it is evident that PCL had a burst release, showing increased NGF release as of day 1 and day 4, and reduced NGF over days 14-28. In contrast, the PCL-BSA nanofibers showed continued release over days 14-28, with at least 50 ng/ml released at all time points over the 28 day period. Further, the cumulative release profile indicates controlled and continuous release from PCL-BSA as compared to the PCL fibers.

NGF was incorporated into electrospun nanofibers of PCL and BSA blends. The release of NGF from fibers was more effective from PCL-BSA blends as compared to plain PCL. The incorporation of BSA appeared to aid in the increased loading and the controlled release over the 28-day time periods, which was absent in the case of plain PCL based matrices. The released NGF still retained bioactivity, as shown by the stimulation of neurite extensions from the PC12 cells.

### Example 7

### Functionalization of the Inner Nanofibrous Spiral Scaffolds by Incorporation of Cells Using Cell Sheets

Poly (N-isopropyl acrylamide) (PNIPAAm) may be utilized to generate temperature responsive surfaces or brushes to control protein and cell interactions. An electrostatic interaction-based deposition of PNIPAAm was used to coat a surface to allow temperature sensitive cell attachment and removal.

### Example 7.1

### Preparation of Temperature-Responsive Multilayer Films

Multilayer films were prepared by alternating deposition of tannic acid (TA) and poly (N-isopropyl acrylamide) (PNIPAAm) onto polyethylene-imine (PEI)/PLL coated 6-well plates. The PEI/PLL, PNIPPAm and TA were sterilized by autoclave prior to deposition. Subsequent deposition techniques were performed aseptically. The layers were deposited for 5 minutes each, then washed in sterile PBS to remove unattached polymers. After deposition of 5 bilayers, the surfaces were washed thrice in sterile PBS, then rinsed in DMEM (2 ml) for 10 minutes.

### Example 7.2

### Cell Culture and Growth of Cell Sheets From Temperature-Responsive Substrates

Osteoblast cells were maintained in a humidified atmosphere in an incubator (37° C.) in phenol red-free DMEM supplemented with 10% fetal bovine serum (FBS) and 1% penicillin streptomycin. Cells were trypsinized, then added (10,000 cells/0.5 ml media) to each well of a 6-well plate prepared in Example 7.1, with the final volume of each well brought to 2 ml with media. The 6-well plate was incubated for about 8 days (or until sufficient confluence of cells was achieved), then transferred to an environment at 4° C. for 30 minutes until the cell sheet detached from the surface of the multilayers.

The total number of cells on the cell sheet was quantified based on extrapolation of the overall surface area of a well in a 6-well plate. The cells sheets then were transferred to the surface of a thin sheet of nanofibrous porous PCL scaffold. The sheet containing the cells then was wrapped to form a spiral shape and incubated in a 24-well plate supplemented with medium (2 ml).

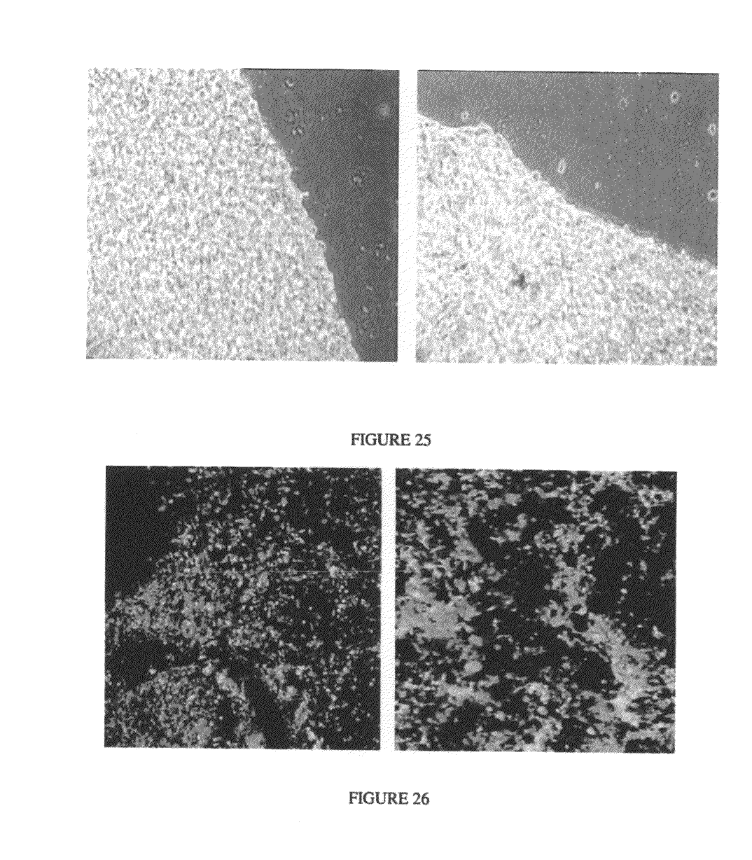

The uniformity of cell loading on the scaffold and the number of viable cells on the scaffolds was microscopically analyzed. FIG. 25 shows photomicrographs of the fabricated cell sheets. The cell sheet formed a uniform layer; which detached in small sections (up to 1 cm×1 cm).

### Example 7.3

### Transfer of Cell Sheets to Porous PCL Scaffolds (Thin Films)

FIG. 26 shows a live-dead image of osteoblast cells on PCL porous sheets. Analysis indicated that a large number of cells were uniformly transferred to the scaffolds, while maintaining viability, to provide high surface coverage. Further, the cells' extracellular matrix (ECM) also was transferred.

### Example 7.4

### Cell Attachment and Proliferation on PCL Sheets

Fabricated cell sheets, once detached from the surface of the multilayers, were suspended in medium. The PCL scaffold was moved under the cell sheet and the entire cell sheet was lifted and transferred to a 12-well plate along with the PCL scaffold. The cell sheet was allowed to attach to the surface for 2 hours, then 2 ml differentiation medium was added onto the scaffolds. The differentiation medium contained the same basal medium (as described above) supplemented with 10 mM β-glycerophosphate (Sigma), 100 nM dexamethasone and 50 μg/ml of ascorbic acid.

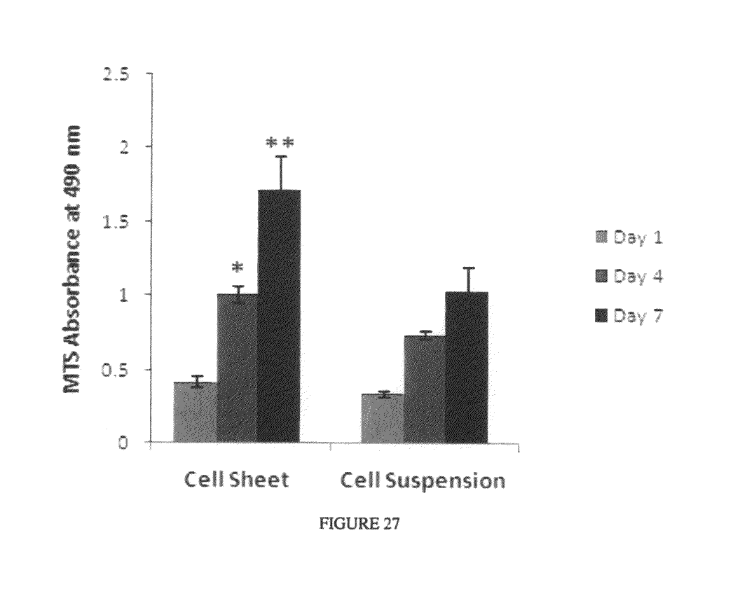

Quantification of cell numbers was performed by MTS assay utilizing a piece (0.5 mm×0.5 mm) of the cell sheet. An equivalent number of trypsinized osteoblast cells (determined with a hemacytometer) served as a standard control. These cells were added to a PCL scaffold. This value was normalized by adding an equivalent amount of cells to untreated tissue culture 6-well plates. This standard control was used for quantification of cells on scaffolds populated with a cell sheet, cells during cell attachment, cell proliferation and determining the osteoblast phenotype at day 1, day 4 and day 7. Samples were analyzed in triplicate to determine the statistical significance. FIG. 27 is a bar chart of MTS absorbance (490 nm) of TCPS, cell sheets and the cell suspension of a 7-day culture. The “*” indicates statistically significant (p<0.05) difference in cell proliferation at day 4 on cell sheet based approach versus cells in suspension; “**” indicates statistically significant (p<0.05) difference in cell attachment between cell sheet based approach and cells in suspension as of day 7. Error bars indicate 3 standard deviations. It appears that a higher number of cells attached to the scaffolds from the cell sheet as compared to cells seeded in suspension. The cell numbers obtained on day 1 through may be indicative of the cell seeding efficiency of both approaches. Without being limited by theory, it may be that the TCPS had the maximum number of cells due to increased surface area as compared to PCL-based scaffolds. Further, on PCL-based scaffolds the cell sheet approach to populate scaffold showed increased cell attachment as compared to the suspension based approach.

### Example 7.5

### Osteoblast Differentiation of PCL Sheets

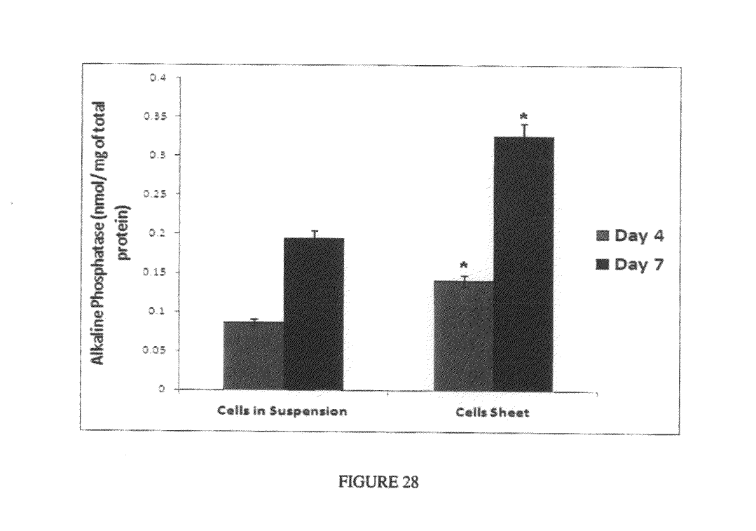

The differentiation capability of cells was evaluated by colorimetric assay for ALP activity. FIG. 28 is a bar chart of ALP activity (nmol/mg of total protein) of the cells in suspension and cell sheets during a 7 day culture. The “*” indicates statistically significant (p<0.05) increase in ALP activity of cell sheet populated scaffold versus suspension populated scaffolds. Error bars indicate 3 standard deviations. The extent of expression of ALP on scaffolds seeded with cell sheets showed increased activity as compared to the scaffolds seeded with cells in suspension at the same time points. These results suggest that increased cell number originating from the cell sheet may enhance cell phenotype expression. From the data it is also evident that the rate of increase of ALP activity on scaffolds seeded with cell sheets is increased when compared to cells seeded in suspension. Without being limited by theory, since ALP is used as an initial differentiation marker, the faster attachment tendency and the increased proliferation rates at early stages supported cell differentiation on scaffolds.

It should be understood that the embodiments described herein are merely exemplary and that a person skilled in the art may make many variations and modifications thereto without departing from the spirit and scope of the present invention. All such variations and modifications, including those discussed above, are intended to be included within the scope of the invention, which is described, in part, in the claims presented below.

What is claimed is:

1. An integrated scaffold for bone tissue engineering having a tubular outer shell formed of at least a first biodegradable polymer and defining a bore having a bore surface; and a spiral scaffold insert including a porous sheet formed of at least a second biodegradable polymer, said porous sheet being wound about an axis such that said porous sheet forms a series of coils about said axis and defines a spiral gap between said series of coils, one of said coils being an outermost coil and having an outer surface, wherein said spiral scaffold insert resides at least partially within said bore of said tubular outer shell,
the improvement comprising at least a portion of said outer surface of said outermost coil integrated with said bore surface such as to provide geometric stability to said spiral scaffold insert.

2. The integrated scaffold of claim 1, said improvement further comprising a mesh of nanofibers deposited on the porous sheet to a depth sufficient to promote cell attachment and proliferation on said spiral scaffold insert.

3. The integrated scaffold of claim 2, wherein said nanofibers include a third biodegradable polymer that is different than said second biodegradable polymer.

4. The integrated scaffold of claim 2, wherein either one or both of said porous sheet and said nanofibers includes an active agent.

5. The integrated scaffold of claim 4, wherein said active agent is a drug or a growth factor.

6. The integrated scaffold of claim 1, said improvement further comprising a stack of bilayers, each bilayer consisting of a polymeric layer including a third polymer and a ceramic layer including a ceramic, said stack arranged such that one of said polymeric layers is attached to said porous membrane through electrostatic attraction and such that each of the other of said polymeric layers is attached to the ceramic layer of an adjacent bilayer through electrostatic attraction.

7. The integrated scaffold of claim 6, wherein said stack of bilayers includes an active agent.

8. The integrated scaffold of claim 7, wherein said active agent is a growth factor or an extracellular matrix protein.

9. The integrated scaffold of claim 1, said improvement further comprising a cell sheet attached to said porous sheet.

10. The integrated scaffold of claim 9, wherein said cell sheet contains only one type of cell.

11. The integrated scaffold of claim 9, wherein said cell sheet contains a combination of cell types selected to produce bone growth and vascularization.

12. The integrated scaffold of claim 1, wherein said tubular outer shell exhibits a Young's modulus and compressive strength similar to those of trabecular bone.

13. A method of making an integrated scaffold for bone tissue engineering including the steps of:
forming a tubular outer shell of at least a first biodegradable polymer such that the tubular outer shell defines a bore having a bore diameter and a bore surface; and
forming a spiral scaffold insert, said step of forming a spiral scaffold insert including the steps of (a) preparing a porous sheet of a biodegradable polymer, (b) placing a sheet of a deformable material on said porous sheet and rolling the sheet of deformable material and the porous sheet about an axis such as to form a spiral structure having alternating coils of the porous sheet and the deformable material and an outer diameter that is approximately as large as the bore diameter of the tubular outer shell, (c) fixing the shape of the spiral structure by performing the steps of heating the spiral structure then freezing the spiral structure, (d) removing the sheet of deformable material from the spiral structure such that said porous sheet defines a spiral gap between the coils of the porous sheet, whereby said spiral insert has an outermost coil formed of said porous sheet and having an outer surface;
the improvement comprising the steps of:
(e) inserting the spiral scaffold insert into the bore of the tubular outer shell such that the outer surface of the outermost coil of the spiral scaffold insert contacts the bore surface, thereby forming an interface between the outer surface of the outermost coil and the bore surface;
(f) applying a solvent at the interface such as to soften the first and second biodegradable polymers at the interface; and
(g) evaporating the solvent such that the outer surface of the outermost coil becomes integrated with the bore surface through interaction of the first and second polymers, thereby providing geometric stability to the spiral scaffold insert.

14. The method of claim 13, the improvement including the further step (h) of depositing a mesh of nanofibers on said porous sheet by electrospinning so as to promof nanofibers deposited on the porous sheet to a depth sufficient to promote cell attachment and proliferation on said spiral scaffold insert, wherein said step (h) is performed before step (b).

15. The method of claim 13, said improvement including the further steps of:
(h) applying a first solution including a third polymer to the porous sheet so as to form a polymeric layer which includes the third polymer and which is attached to the porous sheet through electrostatic attraction;
(i) applying a second solution including a ceramic to the polymeric layer so as to form a bilayer consisting of the polymeric layer and a ceramic layer which includes the ceramic and which is attached to the polymeric layer through electrostatic attraction;
(j) applying the first solution to the ceramic layer of the bilayer so as to form another polymeric layer which is attached to the ceramic layer by electrostatic attraction; and
(k) applying the second solution to the another polymeric layer so as to form another bilayer consisting of the another polymeric layer and another ceramic layer, thereby forming a stack of bilayers on the porous sheet.

16. The method of claim 13, said improvement comprising the further steps of:
(h) aseptically depositing a first sterile solution including tannic acid onto a sterile substrate so as to form a tannic acid layer including tannic acid;
(i) aseptically depositing a second sterile solution including poly(N-isopropyl acrylamide) onto the tannic acid layer so as to form a bilayer consisting of the tannic acid layer and a polymeric layer including poly(N-isopropyl acrylamide);
(j) aseptically depositing the first sterile solution onto the polymeric layer so as to form another tannic acid layer;
(k) aseptically depositing the second sterile solution onto the another tannic acid layer so as to form another bilayer consisting of the another tannic acid layer and another polymeric layer, thereby forming a stack of bilayers on the sterile substrate;
(l) washing the stack of bilayers with a sterile phosphate buffered saline solution and a cell growth medium;
(m) culturing cells on the stack of bilayers so as to form a cell sheet;
(n) removing the cell sheet from the stack of bilayers; and
(o) transferring the cell sheet to the porous membrane, wherein each of said steps (h) through (o) is performed before step (b).
